(19) United States
(12) Patent Application Publication
(10) Pub. No.: US 2024/0393319 A1
(43) Pub. Date: Nov. 28, 2024

(54) FLUORESCENT SENSORS AND METHODS OF MAKING AND USING

(71) Applicants: Howard Hughes Medical Institute, Chevy Chase, MD (US); Weill Cornell Medicine, New York, NY (US)

(72) Inventors: Jonathan S. Marvin, Arlington, VA (US); Tim Brown, New York, NY (US); Tim Ryan, New York, NY (US)

(21) Appl. No.: 18/647,657

(22) Filed: Apr. 26, 2024

Related U.S. Application Data
(60) Provisional application No. 63/462,413, filed on Apr. 27, 2023.
(60) Provisional application No. 63/540,643, filed on Sep. 26, 2023.

(51) Int. Cl. G01N 33/50; C12N 15/86

(52) U.S. Cl. CPC G01N 33/5023; C12N 15/86; C12N 2750/14143

(57) ABSTRACT

This disclosure describes fluorescence-based sensors for detecting and quantifying ATP, and methods of using such sensors in vitro and in vivo.

### CROSS REFERENCE TO RELATED APPLICATIONS

This application claims the benefit of priority under 35 U.S.C. § 119 (e) to U.S. Application No. 63/462,413 filed Apr. 27, 2023, and U.S. Application No. 63/540,643 filed Sep. 26, 2023. These documents are incorporated by reference in their entirety.

### TECHNICAL FIELD

This disclosure generally relates to fluorescent sensors and methods of making and using such fluorescent sensors.

### BACKGROUND

ATP is the universal energy currency in biology, used at every level of cellular function. A number of technologies have been developed for measuring ATP consumption. Unfortunately, each of these sensors are lacking in some respect. The sensors described herein are intended to address these issues and provide a number of benefits.

### SUMMARY

Fluorescence-based sensors for detecting and quantifying ATP are described herein, as are methods of making and using such sensors. Here we report the improvement of iATPSnFR1. iATPSnFR2 has much higher dynamic range (ΔF/F˜12), is available as three different affinity variants, and is fused to spectrally separable fluorescent tags (HaloTag with synthetic far-red fluorophores or mIRFP670nano3), thus providing an approach to normalize the signal to the expression level of the sensor and allowing quantitative comparisons across individual cells or subcellular locations. We show that iATPSnFR2 can provide detailed measurements of the variations in resting ATP values across synapses as well as the kinetics of ATP changes during metabolic perturbations at the cytosolic, single synapse, and single mitochondrial levels. These data show for the first time that individual synapses behave as semi-independent metabolic units, as during metabolic stress, the kinetics of ATP depletion varied significantly even within the same axon.

In one aspect, an ATP-detecting fluorescence-based sensor having at least 95% sequence identity to an amino acid sequence selected from SEQ ID NOs: 1, 3, 5, 7, 9, 11, and 13 is provided.

In some embodiments, the ATP-detecting fluorescence-based sensor has at least 96% sequence identity to an amino acid sequence selected from SEQ ID NOs: 1, 3, 5, 7, 9, 11, and 13. In some embodiments, the ATP-detecting fluorescence-based sensor has at least 97% sequence identity to an amino acid sequence selected from SEQ ID NOs: 1, 3, 5, 7, 9, 11, and 13. In some embodiments, the ATP-detecting fluorescence-based sensor has at least 98% sequence identity to an amino acid sequence selected from SEQ ID NOs: 1, 3, 5, 7, 9, 11, and 13. In some embodiments, the ATP-detecting fluorescence-based sensor has at least 99% sequence identity to an amino acid sequence selected from SEQ ID NOs: 1, 3, 5, 7, 9, 11, and 13. In some embodiments, the ATP-detecting fluorescence-based sensor has 100% sequence identity to an amino acid sequence selected from SEQ ID NOs: 1, 3, 5, 7, 9, 11, and 13.

In yet another aspect, an ATP-detecting fluorescence-based sensor having at least 95% sequence identity to the amino acid sequence shown in SEQ ID NO: 13 and including at least A95K and S29W substitutions is provided.

In still another aspect, an ATP-detecting fluorescence-based sensor having at least 95% sequence identity to the amino acid sequence shown in SEQ ID NO: 13 and including at least an A95K substitution is provided.

In another aspect, an ATP-detecting fluorescence-based sensor having at least 95% sequence identity to the amino acid sequence shown in SEQ ID NO: 13 and including at least A95A and A119L substitutions is provided.

In still another aspect, a nucleic acid encoding any of the ATP-detecting fluorescence-based sensors described herein is provided.

In yet another aspect, a vector including the nucleic acid encoding any of the ATP-detecting fluorescence-based sensors described herein is provided.

In still another aspect, a cell is provided that includes any of the nucleic acids described herein or any of the vectors described herein.

In another aspect, methods of determining the amount of ATP in a cell are provided. Such methods typically include providing a cell including any of the ATP-detecting fluorescence-based sensor as described herein and determining the amount of fluorescence emitted from the sensor.

In some embodiments, the method further includes exposing the cell to ATP or a source thereof. In some embodiments, the cell expresses a nucleic acid as described herein. In some embodiments, the cell includes a vector as described herein.

In some embodiments, the cell is in vitro. In some embodiments, the cell is in vivo.

In yet another aspect, an article of manufacture is provided. Such an article of manufacture can include any of the ATP-detecting fluorescence-based sensors described herein, any of the nucleic acids described herein, or any of the vectors described herein.

In some embodiments, the article of manufacture further includes ATP. In some embodiments, the article of manufacture further includes an ATP inhibitor. In some embodiments, the article of manufacture further includes instructions for using the ATP-detecting fluorescence-based sensor.

In one aspect, an ATP-detecting fluorescence-based sensor having at least 95% sequence identity (e.g., at least 96%, 97%, 98%, 99% or 100% sequence identity) to the amino acid sequence shown in SEQ ID NO: 13 is provided. In another aspect, an ATP-detecting fluorescence-based sensor having at least 95% sequence identity (e.g., at least 96%, 97%, 98%, 99% or 100% sequence identity) to the amino acid sequence shown in SEQ ID NO: 13 and including at least A95K and S29W substitutions is provided. In still another aspect, an ATP-detecting fluorescence-based sensor having at least 95% sequence identity (e.g., at least 96%, 97%, 98%, 99% or 100% sequence identity) to the amino acid sequence shown in SEQ ID NO:13 and including at least an A95K substitution is provided. In yet another aspect, an ATP-detecting fluorescence-based sensor having at least 95% sequence identity (e.g., at least 96%, 97%, 98%, 99% or 100% sequence identity) to the amino acid sequence shown in SEQ ID NO:13 and including at least A95A and A119L substitutions is provided.

In one aspect, a nucleic acid encoding the ATP-detecting fluorescence-based sensor described herein is provided. In some embodiments, the nucleic acid comprises at least 95% sequence identity (e.g., at least 96%, 97%, 98%, 99% or 100% sequence identity) to a nucleic acid sequence selected from SEQ ID NOs: 2, 4, 6, 8, 10, 12 and 14.

In another aspect, a vector including the nucleic acid encoding the ATP-detecting fluorescence-based sensor described herein is provided. In another aspect, a cell comprising the nucleic acid or the vector is provided.

In one aspect, methods of determining the amount of ATP in a cell are provided. Such methods typically include providing a cell comprising the ATP-detecting fluorescence-based sensor as described herein and determining the amount of fluorescence emitted from the sensor. In some embodiments, the method further includes exposing the cell to ATP or a source thereof.

In some embodiments, the cell expresses the nucleic acid described herein. In some embodiments, the cell includes the vector described herein. In some embodiments, the cell is in vitro. In some embodiments, the cell is in vivo.

In another aspect, an article of manufacture is provided. Such an article of manufacture can include an ATP-detecting fluorescence-based sensor as described herein, a nucleic acid encoding the sensor as described herein, or a vector that includes a nucleic acid encoding the sensor as described herein. In some embodiments, the article of manufacture further includes ATP. In some embodiments, the article of manufacture further includes an ATP inhibitor. In some embodiments, the article of manufacture further includes instructions for using the ATP-detecting fluorescence-based sensor.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1E shows the design and characterization of iATPSnFR2 in vitro and in fibroblasts. (1A) Artistic rendering of iATPSnFR2.HaloTag made with PyMol. The N-terminal fragment (residues 1-109) of the epsilon subunit of ATPase (orange, based on 2E5Y.PDB) is fused to cpSFGFP (green). The C-terminus of cpSFGFP is fused to the residual ATP-binding helix (residues 110-129) of the epsilon subunit (pink), which itself is fused to HaloTag (cyan). “Linkers” are shown as sticks in yellow. ATP is shown as sticks in black. HaloTag fluorophore is shown as sticks in red. (1B) iATPSnFR2.HaloTag-JFX650 affinity variants titrated with ATP. Grey, S29W.A95K (4 μM); orange, A95K (16 μM), yellow, A95A.A119L (530 μM). (1C-1E) Ratio of green (iATPSnFR2) to red (HaloTag-JFX650) fluorescence in fibroblasts transfected with three affinity variants of iATPSnFR2-HaloTag. Grey, S29W.A95K (high affinity); orange, A95K (medium affinity); yellow, A95A.A119L (low affinity). Cells were incubated for 21 minutes in buffer containing 10 mM glucose, and then switched to buffer containing 10 mM 2-deoxyglucose. (1C) Rho° cells. (1D) Parental LMTK cell line. (1E) Parental cell line treated with both 2-deoxyglucose and oligomycin.

FIG. 2 shows the response of iATPSnFR2 in matrix of axonal mitochondria to glycolytic and mitochondrial ATP synthase inhibitors. iATPSnFR2.A95A.A119L.HaloTag targeted to mitochondria with 4×-COX8 signal sequence was imaged for HaloTag-JF635 in 5 mM glucose. iATPSnFR2.A95A.A119L was imaged with perfusion of buffers containing: 5 mM glucose, 5 mM glucose+10 μM koningic acid (KA), 1.25 mM lactate+1.25 mM pyruvate, and finally 1.25 mM lactate+1.25 mM pyruvate+10 μM oligomycin. Scale bar: 10 μm. Data is represented as mean±S.E. Images at top align with treatments and trace below. JF635 channel (red image) remained stable throughout the experiment.

FIG. 3 shows the depletion of cytosolic ATP in nerve terminals during action potential firing. Bottom left: Ratio of green to red fluorescence of axonal terminals in cultured neurons expressing iATPSnFR2.HaloTag-JFX650 during a burst of AP firing (6 sec at 50 Hz (grey bar)) in either 5 mM glucose (dashed lines) or 5 mM glucose+10 μM koningic acid (solid lines). Low affinity variant A95A.A119L in yellow, medium affinity variant A95K in orange. Identical experiments carried out using cpSFGFP control (green) showed no change for the same stimulus for either condition. Top: images at the completion of the experiment in both green and red channels; scale bar 10 μm. Bottom Right: Maximum change in ratio during the stimulation period in the absence of KA (open circles) or presence of 10 μM KA (filled circles).

FIG. 4A-4F shows the spontaneous depletion of ATP in boutons detected by iATPSnFR2. iATPSnFR2 was targeted to boutons by fusing it to the C-terminal of synaptophysin. (4A) Average ratio of green to red fluorescence of iATPSnFR2-HaloTag-JFX650 normalized to pre-treatment baseline. Yellow, low affinity variant A95A.A119L (n=7); orange, medium affinity variant A95K (n=6), +s.c. Treatment with 5 mM glucose+10 μM KA+0.3 μM TTX initiated at t=0. (4B) Variation in ATP across individual boutons and across individual neurons pre- and post-ATP depletion by koningic acid of the 7 cells shown in 4A) with the low affinity variant. (4C) Representative raw traces from individual boutons expressing A95A.A119L variant. (4D) Normalization of representative traces illustrate that individual boutons have unique half-times (t_{1/2}) to ATP depletion and unique rates of ATP consumption. (4E) Cross correlation of single bouton depletion rates versus t_{1/2} values shows there is in general an inverse relationship between these parameters, with some cells occupying regions of the parameter space. (4F) Boutons from only two cells (cells 4 & 7 in FIG. 4B) shown for clarity.

FIG. 5A shows in vitro characterization of iATPSnFR2 affinity for ATP and decoys. Three variants of iATPSnFR2.HaloTag-JFX650 titrated with ATP (black) and ADP (grey), head-to-head. S29W.A95K (left); A95K (center); A95A.A119L (right). Binding curve fits, in μM, for ATP and ADP are S29W.A95K: 4 μM, 400 μM. A95K: 16 μM, 780 μM. A95A.A119L: 530 μM, 5300 μM.

FIG. 5B shows in vitro characterization of the excitation spectra of iATPSnFR2.S29W (grey), iATPSnFR2.A95K (orange), iATPSnFR2.A95A.A119L (yellow), and cpSFGFP (green) in the absence of ATP (left, dashed lines) and in the presence of 7 mM ATP. Emission observed at 515 nm (5 nm bandpass). Excitation scanned with 5 nm bandpass. Inset shows A95A.A119L variant spectra±ATP to indicate the isosbestic point.

FIG. 5C shows in vitro characterization of the emission spectra of iATPSnFR2.S29W (grey), iATPSnFR2.A95K (orange), iATPSnFR2.A95A.A119L (yellow), and cpSFGFP (green) in the absence of ATP (left, dashed lines) and in the presence of 7 mM ATP. Excitation at 485 nm (5 nm bandpass). Emission scanned with 5 nm bandpass.

FIG. 5D shows in vitro characterization of iATPSnFR2 affinity for ATP and decoys. Three variants of iATPSnFR2.HaloTag-JFX650 titrated with ATP (black) and ADP (grey), head-to-head. S29W.A95K (left); A95K (center); A95A.A119L (right). Binding curve fits, in μM, for ATP and ADP are S29W.A95K: 4 μM, 400 μM. A95K: 16 μM, 780 μM. A95A.A119L: 530 μM, 5300 μM.

FIG. 5E shows in vitro characterization of iATPSnFR2 affinity for ATP and other nucleoside triphosphates. Three variants of iATPSnFR2.HaloTag-JFX650 titrated with ATP and other NTPs, head-to-head. S29W.A95K (left); A95K (center); A95A.A119L (right). ATP (black), CTP (cyan), GTP (grey), TTP (purple). Binding curve fits, in μM, for ATP, CTP, GTP are S29W.A95K: 20 μM, 50 μM, 430 μM. A95K: 30 μM, 100 μM, 1500 μM. A95A.A119L: 750 UM, ˜10 mM, ˜10 mM.

FIG. 5F shows in vitro characterization of the decoys. The A95A.A119L.HaloTag-JFX650 sensor is negligibly affected by inorganic phosphate. Head-to-head titration of ATP without added phosphate (black) and with 20 mM PO4 (grey) (diluted from 1 M NaPO4 stock, pH 7).

FIG. 5G shows in vitro characterization of the temperature dependence. ATP titration of iATPSnFR2.A95A.A119L variant at 25° C. (grey) and 37° C. (black). Error bars are s.d. of three technical replicates. Fluorescence was measured in a Cytation 5 plate reader, which has relatively rapid heating.

FIG. 5H shows in vitro characterization of the kinetics. The three different variants of iATPSnFR2 bind ATP within a one or two seconds of rapid mixing. S29W.A95K (left) and A95K (center) bind ATP faster than A95A.A119L (right). The data from stopped flow fluorescence do not fit to a single exponential (not shown), but very well to the addition of two exponentials (shown), indicating a more complicated, three-state mechanism of binding and fluorescence change. Panels are the sensor with a C-terminal fusion to HaloTag-JFX650. Final concentrations of ATP are: 10000, 3333, 1111, 370, 123 μM. Fluorescence data collected on an Applied Photophysics SX-20 stopped-flow apparatus with 490 nm LED excitation and 525 nm long pass filter. Data points are average of 5 technical replicates.

FIG. 5I shows in vitro characterization of pH dependence. iATPSnFR2.A95A.A119L.HaloTag and cpSFGFP were diluted to 0.2 μM in Mammalian Cell Imaging Buffer at variable pH. Fluorescence was measured at Ex/Em 485 nm/535 nm (20 nm bandpass) or Ex/Em 600 nm/630 nm (10 nm bandpass) without ATP (dashed lines). Then ATP was added to 2.5 mM from a concentrated stock and fluorescence was remeasured (solid lines). Each plot is a different presentation of the same data. In the top left panel, the fluorescence of cpSFGFP has been adjusted to approximately match that of ligand-free state of the sensor at pH 7.4. In the top right panel, the fluorescence of cpSFGFP has been adjusted to match that of the ATP-bound state of the sensor at pH 7.4. In the lower left plot, each data set has been adjusted so that its maximum value is 1. (Black, iATPSnFR2 green channel; green, cpSFGFP green signal).

FIG. 6 shows the validation in fibroblast cell culture. Left: Ratio of green to red (HaloTag-JFX650) fluorescence in p⁰ cells transfected with iATPSnFR2.A95A.A119L variant (yellow) or cpSFGFP (green). Buffer containing 10 mM glucose was refreshed twice (at the points where fluorescence increased). Right: Same as on left, but with a switch from 10 mM glucose to 10 mM 2-deoxyglucose, and then back to 10 mM glucose.

FIG. 7A shows the validation of mito-iATPSnFR2.HaloTag in neuronal culture. Fluorescence traces of both green (iATPSnFR2) and red (HaloTag-JF635) channels in neurons expressing mito-iATPSnFR2.A95A.A119L.HaloTag in mitochondria during perfusion with glucose and KA. Shown is the average fluorescence traces from 4 neurons (+/−SEM), with each neurons proving signals form 40-50 axonal mitochondria.

FIG. 7B shows the validation of mito-iATPSnFR2.miRFP670nano3 in neuronal culture. Average fluorescence traces (n=4) of both green (iATPSnFR2) and red (mIRFP670nano3) channels neurons expressing mito-iATPSnFR2.A95A.A119L.mIRFP670nano3 during perfusion with glucose and glucose with KA.

FIG. 8A shows the validation of Syn-iATPSnFR2.HaloTag in axonal boutons. Fluorescence traces of both green (iATPSnFR2 and red (HaloTag-JF635 or HaloTag-JFX650) channels in neurons expressing syn-iATPSnFR2.A95A.A119L.HaloTag on the cytosol-facing surface of synaptically targeted vesicles during perfusion with glucose and KA.

FIG. 8B shows in vitro titration of HaloTag-JF635 with ATP. Fluorescence of purified HaloTag-JF635 (0.2 μM in Mammalian Cell Imaging Buffer) was measured (Ex 625 nm, Em 670 nm, 20 nm bandpass) with varying concentration of ATP.

FIG. 9A-9H are data showing that synaptic PGK1 expression confers synaptic resilience under hypometabolic stress. (9A) Ensemble average vGlutI-pH fluorescence in neurons expressing PGK1-HALO (red) shows that synaptic endurance is fully restored in 0.1 mM glucose compared to controls (blue). (9B) The remaining fluorescence 55 s post stimulation after each train is plotted as mean±SEM, control N=23, PGK1-HALO N=12*p<0.05, ***p<0.001 2-way ANOVA. (9C) PGK1 (magenta) and synapsin (red) immunofluorescence shows that PGK1 is present in nerve terminals (white arrows). (9D) PGK1-HALO (labelled with JF585) also accumulates in nerve terminals (vGlutI-pH, visualized during NH4Cl application) scale bar in 9C and 9D, 6 μm. (9E) Synaptic endurance score, measured as the fluorescence signal of recovery after the 10th round for each cell tested compared to the average synaptic PGK1-HALO expression normalized to non-transfected cells N=12. Dashed blue line shows the average synaptic endurance score for low glucose in control neurons after the 10th round. (9F) Schematic of the synapto-iATPSnFR2-miRFP670nano3 sensor used for synaptic ATP measurements. (9G) Ensemble average synapto-iATPSnFR2-miRFP670nano3 traces for control (blue) and PGK1-HALO (red) transfected cells stimulated with 600 APs at 10 Hz in 0.1 mM glucose. PGK1-HALO neurons show a significant activity dependent upregulation of ATP synthesis following activity. (9H) Comparison of absolute nerve terminal ATP values pre-stimulus (left) at the end of the stimulus (left dotted line in (PGK1-HALO)) (middle) and 35 s post-stimulus (right dotted line in 9C) (right) in control and PGK1-HALO expressing neurons, mean±SEM indicated, control N=12, PGK1-Halo N=11. *p<0.05, **p<0.01 unpaired t-test.

FIG. 10A-10D are experimental data showing that terazosin confers metabolic synaptic resilience. (10A) Ensemble average vGlutI-pH traces for control (blue) and Terazosin (TZ) treated (green) primary hippocampal neurons subjected to repeated stimulation in 0.1 mM glucose (TZ chemical structure shown in window). (10B) The synaptic endurance, measured as the remaining fluorescence 55 sec after each AP bout (mean±SEM) for the traces in (10A) show that TZ confers significant resilience. Control N=23, Terazosin N=10*p<0.05, **p<0.01 2-way ANOVA. (10C) The kinetics of presynaptic ATP measured with synapto-iATPSnFR2-miRFP670nano3, normalized to the pre-stimulus values reveal that TZ incubated cells stimulated with 600 APs in 0.1 mM glucose have significantly smaller activity-induced drops in ATP and more rapid post-stimulus recovery as well as higher starting ATP. (10D) Comparison of the absolute nerve terminal ATP values pre-stimulus (left) at the end of the stimulus (middle) and 35 s post-stimulus (right) in control and TZ treated neurons mean±SEM indicated, control N=12, TZ N=17. *p<0.05, **p<0.01 unpaired t-test.

FIG. 11A-11H are experimental results showing that PARK7/DJ-1 is necessary for PGK1 driven hypometabolic resilience. (11A) Ensemble vGlutI-pH traces in neurons, expressing PGK1-HALO (red) or PGK1-HALO with DJ-1 KD (black) and (11C) TZ treated neurons (green) versus TZ treated in DJ-1 KD (black) subjected to repeated electrical stimulation (11B) Synaptic endurance measured vGlutI-pH fluorescence 55 s after each stimulus bout for the traces in (11A) mean±SEM, PGK1-HALO N=12, PGK1-HALO+DJ-1 KD N=7**p<0.01, ***p<0.001 2-way ANOVA and (11D) mean±SEM, Terazosin N=10, Terazosin+DJ-1 KD N=10*p<0.05, ***p<0.001 mixed-effects (11E) The kinetics of presynaptic ATP normalized to the pre-stimulus values when neurons were stimulated with 600 APs at 10 Hz in 0.1 mM glucose in absence of DJ-1 reveal a significantly larger activity-induced drop in ATP and defective post-stimulus recovery. (11F) Comparison absolute nerve terminal ATP values pre-stimulus (left) at the end of the stimulus (middle) and 35 s post-stimulus (right) in control and DJ-1 KD neurons mean±SEM indicated, control N=12, DJ-1 KD N=11. *p<0.05, ***p<0.001 unpaired t-test. (11G) The kinetics of presynaptic ATP normalized to the pre-stimulus values when neurons were stimulated with 600 APs at 10 Hz in 0.1 mM glucose in TZ and DJ-1 KD reveal an inability of TZ to accelerate ATP kinetics in absence of DJ-1. (11H) Comparison absolute nerve terminal ATP values pre-stimulus (left) at the end of the stimulus (middle) and 35 s post-stimulus (right) in TZ and TZ with DJ-1 KD neurons mean±SEM indicated, TZ N=17, TZ DJ-1 KD N=8. *p<0.05, ***p<0.001 unpaired t-test.

FIG. 12A-12C are experimental results showing synaptic activity and CPT1 facilitate FA transfer to mitochondrial matrix to maintain ATP level in absence of glucose. (12A) Schematic illustration of C-terminally Halo-tagged ATP sensor (ATPsnFR2) targeted into mitochondrial matrix with the help of four repeats of N-terminal leader sequence, mito. (12B) ATP levels (normalized using JF635) in the mito-ATPsnFR2-Halo expressing hippocampal neurons in absence of any fuel, PA induced or CPT1 inhibited condition (by Etomoxir) to the PA induced neurons. Data is represented as mean±SE for N=4 (each condition). (12C) Relative change (in %) in the ATP levels after 10 mins of perfusion with the media as described in (12B). Data from N=4 for each condition is represented as mean±SE, p-value (**≤0.001 for PA and PA+Etox) was determined using unpaired samples t-test. ns signifies non-significant change in mean values.

### DETAILED DESCRIPTION

ATP is a critical biochemical currency. Its hydrolysis to ADP provides the free energy necessary to drive numerous physiological processes. The importance of ATP is evident by the plethora of routes that exist to convert the energy stored in the form of combustible hydrocarbons into ATP. The essentials of glycolysis and oxidative phosphorylation have been established for over sixty years, having been successfully dissected through in vitro enzymology and biochemistry. One trade-off with this type of reductionism is that it is often hard to recapitulate the physiological milieu in vitro, and therefore the nuances of how molecular pathways are regulated in real time in specific subcellular locations are missed. Fluorescent biosensors are designed to fill the knowledge gap between in vitro reconstitution biochemistry and cellular physiology as they can reveal subcellular dynamics of different metabolites or ions with high temporal and spatial resolution in living cells and tissues. Successful deployment of a fluorescent biosensor requires that the affinity of the sensor matches the physiological concentration of its cognate analyte and that the sensor discriminates against chemically related species. The signal-to-noise ratio is determined by the ratio of the sensor's fluorescence in the bound and unbound states and sets the limit of what magnitude changes in the analyte concentration can be detected in the face of other sources of fluctuation and therefore the spatial scale over which the signal must be averaged. Several promising strategies have emerged in the last ten years to develop a genetically encoded sensor for ATP.

An intensity-based ATP Sensing Fluorescent Reporter, iATPSnFR (Lobas et al., 2019, Nat. Comm., 10:711), was developed by inserting circularly permuted superfolder GFP between the two ATP-binding helices of the epsilon subunit of the F₀-F₁ ATPase of thermostable Bacillus subtilis PS3, which undergoes a large conformational change upon binding ATP. Other research groups have made similarly conceived sensors, albeit with different topologies, ATP affinities, and dynamic ranges. These include ChemoG-ATPSiR, MaLion, and QUEEN, which, like iATPSnFR have a common ancestor in “ATeam”, a FRET based sensor in which CFP and YFP were placed at the N- and C-termini of this bacterial & subunit of F₀-F₁ ATPase, and whose FRET efficiency improves as this subunit changes conformation up binding ATP. These cytosolic ATP sensors have been reviewed recently. Unfortunately, each of these sensors has been lacking in some respect. QUEEN detects changes in ATP by reporting different excitation wavelengths, which is not practically useful for most imaging experiments, especially for preparations other than monolayers. MaLion is available as either a green and or red fluorescent sensor and has almost 5-fold increase in fluorescence upon saturation with ATP, but loses dynamic range at higher temperatures. The most recently developed ATP sensor, ChemoG-ATPSiR, is an improved chemogenetic FRET sensor with 10-fold change in fluorescence ratio. ChemoG-ATPsiR has the necessary large dynamic range, and has an affinity of about 2 mM, which is in the physiological range for many cells. It also is available in different colors, depending on the FRET acceptor used. However, the use of two fluorophores occupies spectral bandwidth that might limit its application to multiplex imaging. Other ATP sensors based on the bacterial ATP regulatory domain GlnK1, including Perceval and PercevalHR, have similar affinities for ATP and ADP, and thus provide a measure of the ratio of ATP: ADP more than the concentration of ATP alone. Finally, firefly luciferase can be used to measure the concentration of ATP, as there is a direct relationship between ATP consumption and light production. This approach is obviously limited in its spatial and temporal resolution but was successfully adapted to observe ATP consumption at nerve terminals in primary dissociated neurons. Even so, that sensor is difficult to deploy due to the low photon flux and limited applicability to modern optical sectioning.

Here, we present an improved ATP sensor-iATPSnFR2, optimized for both higher fluorescence changes in response to saturating ATP (ΔF/F˜12), and modulation of its affinity for ATP such that its range of maximal sensitivity (K_{d}) matches the expected range of ATP concentrations within normally functioning cells, including neurons. We also provide two higher affinity variants to serve as controls or for use in other cell types or cellular sub-compartments that might be expected to have lower concentrations of ATP. We validate its utility for measuring changes in cytosolic ATP using cellular imaging in cultured cell lines, perturbed with different pharmacological compounds known to affect ATP production and consumption. Inclusion of a far-red fluorophore (via HaloTag or mIRFP670nano3) allows for ratiometric measurement to account for expression and movement artefacts. iATPSnFR2 can be targeted to the axonal mitochondria, where it reports homeostasis of mitochondrial ATP in presence of a suitable fuel for the tri-carboxylic acid cycle. It also reports depletion and subsequent rescue of mitochondrial ATP after perfusion of oxphos substrate and inhibitors. Finally, by targeting it to boutons, we can observe spontaneous depletion of ATP and observe that each bouton has its own unique ATP consumption profile.

While multiple other fluorescence-based sensors have been developed, none of them have been widely adopted for determination of ATP consumption in cell culture or in vivo. ATP production pathways through glycolysis produces lactate and acidifies the extracellular space. ATP production from the electron transport chain in mitochondria consumes 02. The extracellular acidification rate (ECAR) and oxygen consumption rate (OCR) can be measured and used as indirect proxies for ATP consumption and are the underlying technologies of the Seahorse analyzer. Typically, these rates are measured in cell culture under normal/healthy conditions to establish a baseline, and then mitochondrial inhibitors (oligomycin and rotenone/antimycin A, but sometimes other inhibitors) are added to drop mitochondrial ATP production to zero, and through a series of complex calculations, ATP production rates, divided between mitochondrial or glycolytic production, are determined.

Measuring pH and O₂ consumption in cell culture is useful for determining the energetic expenditure differences between cell types or between cell cultures that have been treated with different drugs, but the technique also has significant limitations. Primarily, measuring pH changes and O₂ consumption are just proxies for ATP production, and not a direct measurement. Other metabolic events or perturbations that affect pH and O₂ use will be interpreted as ATP consumption. Importantly, the Seahorse system is limited to cell culture analysis and is not compatible with in vivo applications or measurement of ATP consumption with other manipulations, such as electrical stimulation. This technology also affords no spatial resolution either at the single cell or subcellular length scale. Furthermore, once cells have been treated with mitochondrial poisons, they are no longer viable and cannot be assayed again. Finally, the time course of measurement using such devices requires integration of signals over time and is not compatible with sub-second resolution. We expect that iATPSnFR2 will provide researchers with previously exciting new opportunities to study ATP dynamics with temporal and spatial resolution that has, until now, been unavailable.

iATPSnFR Sequences
L1-VLVG.L2-ICV.A95A.A119L
SEQ ID NO: 1
MGRSMKTIHVSVVTPDGPVYEDDVEMVSVKAKSGELGILPGHIPL
KAPLEISAARLKKGGKTQYIAVSGGNLEVRPDKVTIYAQAAERAE
DIDVLRAKAAKERAERRLQSQVLVGNVYITADKQKNGIKANFKIR
HNVEDGSMQLADHYQQNTPIGDGPVLLPDNHYLSTQSVLSKDPNE
KRDHMVLLEFVTAAGITLGMDELYKGGTGGSMSKGEELFTGVVPI
LVELDGDVNGHKFSVRGEGEGDATNGKLTLKFICTTGKLPVPWPT
LVTTLTYGVQCFSRYPDHMKQHDFFKSAMPEGYVQERTISFKDDG
TYKTRAEVKFEGDTLVNRIELKGIDFKEDGNILGHKLEYNFICVD
IDEKRAELLLKRAMNRLSVAEMK
SEQ ID NO: 2
ATGggcAGATCTATGAAGACTATTCACGTGAGTGTCGTAACTCCC
GACGGGCCTGTATATGAGGATGACGTTGAAATGGTGAGCGTCAAA
GCAAAAAGTGGCGAGCTCGGTATTCTCCCAGGCCACATTCCCTTG
AAAGCTCCCCTAGAGATCAGTGCCGCACGCCTGAAGAAAGGGGGC
AAAACACAGtatATCGCTGTGTCAGGCGGCAACCTCGAAGTGCGG
CCTGACAAGGTGACCATCTATGCCCAGGCAGCCGAGAGGGCTGAG
GATATCGATGTCCTGCGCGCCAAGgccGCCAAGGAGAGAGCCGAG
CGCCGACTCCAATCACAGGTCCTGGtCgggAACGTCTATATCACC
GCCGACAAGCAGAAGAACGGCATCAAGGCGAACTTCAAGATCCGC
CACAACGTGGAGGACGGCAGCaTGCAGCTCGCCGACCACTACCAG
CAGAACACCCCCATCGGCGACGGCCCCGTGCTGCTGCCCGACAAC
CACTACCTGAGCACCCAGTCCGTGCTGAGCAAAGACCCtAACGAG
AAGCGCGATCACATGGTCCTGCTGGAGTTCGTGACCGCCGCCGGG
ATCACTCTCGGCATGGACGAGCTGTACAAGGGCGGTACCGGAGGG
AGCATGAGCAAGGGCGAGGAGCTGTTCACCGGGGTGGTGCCCATC
CTGGTCGAGCTGGACGGCGACGTAAACGGCCACAAGTTCAGCGTG
CGCGGCGAGGGCGAGGGCGATGCCACCAACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCAGCTTCAAGGACGACGGC
ACCTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTTTatttgtgtgGAC
ATCGATTTTAAGCGCGCCGAGCTCttgCTTAAGCGCGCAATGAAT
AGGCTCTCAGTTGCCGAAATGAAG
L1-VLVG.L2-ICV.A95A.A119L.Halotag
SEQ ID NO: 3
MGRSMKTIHVSVVTPDGPVYEDDVEMVSVKAKSGELGILPGHIPL
KAPLEISAARLKKGGKTQYIAVSGGNLEVRPDKVTIYAQAAERAE
DIDVLRAKAAKERAERRLQSQVLVGNVYITADKQKNGIKANFKIR
HNVEDGSMQLADHYQQNTPIGDGPVLLPDNHYLSTQSVLSKDPNE
KRDHMVLLEFVTAAGITLGMDELYKGGTGGSMSKGEELFTGVVPI
LVELDGDVNGHKESVRGEGEGDATNGKLTLKFICTTGKLPVPWPT
LVTTLTYGVQCFSRYPDHMKQHDFFKSAMPEGYVQERTISFKDDG
TYKTRAEVKFEGDTLVNRIELKGIDFKEDGNILGHKLEYNFICVD
IDEKRAELLLKRAMNRLSVAEMKLQGGGSGGGGSGGGGSMAEIGT
GFPFDPHYVEVLGERMHYVDVGPRDGTPVLFLHGNPTSSYVWRNI
IPHVAPTHRTIAPDLIGMGKSDKPDLGYFFDDHVREMDAFIEALG
LEEVVLVIHDWGSALGFHWAKRNPERVKGIAFMEFIRPIPTWDEW
PEFARETFQAFRTTDVGRKLIIDQNVFIEGTLPMGVVRPLTEVEM
DHYREPELNPVDREPLWREPNELPIAGEPANIVALVEEYMDWLHQ
SPVPKLLFWGTPGVLIPPAEAARLAKSLPNVKAVDIGPGLNLLQE
DNPDLIGSEIARWLSTLEISG
SEQ ID NO: 4
ATGggcAGATCTATGAAGACTATTCACGTGAGTGTCGTAACTCCC
GACGGGCCTGTATATGAGGATGACGTTGAAATGGTGAGCGTCAAA
GCAAAAAGTGGCGAGCTCGGTATTCTCCCAGGCCACATTCCCTTG
AAAGCTCCCCTAGAGATCAGTGCCGCACGCCTGAAGAAAGGGGGC
AAAACACAGtatATCGCTGTGTCAGGCGGCAACCTCGAAGTGCGG
CCTGACAAGGTGACCATCTATGCCCAGGCAGCCGAGAGGGCTGAG
GATATCGATGTCCTGCGCGCCAAGgccGCCAAGGAGAGAGCCGAG
CGCCGACTCCAATCACAGGTCCTGGtCgggAACGTCTATATCACC
GCCGACAAGCAGAAGAACGGCATCAAGGCGAACTTCAAGATCCGC
CACAACGTGGAGGACGGCAGCaTGCAGCTCGCCGACCACTACCAG
CAGAACACCCCCATCGGCGACGGCCCCGTGCTGCTGCCCGACAAC
CACTACCTGAGCACCCAGTCCGTGCTGAGCAAAGACCCtAACGAG
AAGCGCGATCACATGGTCCTGCTGGAGTTCGTGACCGCCGCCGGG
ATCACTCTCGGCATGGACGAGCTGTACAAGGGCGGTACCGGAGGG
AGCATGAGCAAGGGCGAGGAGCTGTTCACCGGGGTGGTGCCCATC
CTGGTCGAGCTGGACGGCGACGTAAACGGCCACAAGTTCAGCGTG
CGCGGCGAGGGCGAGGGCGATGCCACCAACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCAGCTTCAAGGACGACGGC
ACCTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTTTatttgtgtgGAC
ATCGATTTTAAGCGCGCCGAGCTCttgCTTAAGCGCGCAATGAAT
AGGCTCTCAGTTGCCGAAATGAAGCTGCAGggcggaggctcgggc
ggaggtgggtcgggtggcggcggatcaatggccgagatcggcacc
ggcttccccttcgacccccactacgtggaggtgctgggcgagcgc
atgcactacgttgacgtgggcccccgcgacggcacccccgtgctg
ttcctgcacggcaaccccaccagcagctacgtgtggcgcaacatc
atcccccacgtggcccccacccaccgcactatcgcccccgacctg
atcggcatgggcaagagcgacaagcccgacctgggctacttcttc
gacgaccacgtgcgcttcatggacgccttcatcgaggccctgggc
ctggaggaggtggtgctggtgatccacgactggggcagcgccctg
ggcttccactgggccaagcgcaaccccgagcgcgtgaagggcatc
gccttcatggagttcatccgccccatccccacctgggacgagtgg
cccgagttcgcccgcgagaccttccaggccttccgcaccaccgac
gtgggccgcaagctgatcatcgaccagaacgtgttcatcgagggc
accctgcccatgggcgtggtgcgccccctgaccgaggtggagatg
gaccactaccgcgagcccttcctgaaccccgtggaccgcgagccc
ctgtggcgcttccccaacgagctgcccatcgccggcgagcccgcc
aacatcgtggccctggtggaggagtacatggactggctgcaccag
agccccgtgcccaagctgctgttctggggcacccccggcgtgctg
atcccccccgccgaggccgcccgcctggccaagagcctgcccaac
gttaaggccgtggacatcggccccggcctgaacctgctccaggag
gacaaccccgacctgatcggcagcgagatcgcccgctggctgagc
accctCgagatcagcggc
pAAV.CAG (cyto).L1-VLVG.L2-ICV.A95K.HaloTag
SEQ ID NO: 5
MGRSMKTIHVSVVTPDGPVYEDDVEMVSVKAKSGELGILPGHIPL
KAPLEISAARLKKGGKTQYIAVSGGNLEVRPDKVTIYAQAAERAE
DIDVLRAKKAKERAERRLQSQVLVGNVYITADKQKNGIKANFKIR
HNVEDGSMQLADHYQQNTPIGDGPVLLPDNHYLSTQSVLSKDPNE
KRDHMVLLEFVTAAGITLGMDELYKGGTGGSMSKGEELFTGVVPI
LVELDGDVNGHKFSVRGEGEGDATNGKLTLKFICTTGKLPVPWPT
LVTTLTYGVQCFSRYPDHMKQHDFFKSAMPEGYVQERTISFKDDG
TYKTRAEVKFEGDTLVNRIELKGIDFKEDGNILGHKLEYNFICVD
IDEKRAELALKRAMNRLSVAEMKLQGGGSGGGGSGGGGSMAEIGT
GEPFDPHYVEVLGERMHYVDVGPRDGTPVLFLHGNPTSSYVWRNI
IPHVAPTHRTIAPDLIGMGKSDKPDLGYFFDDHVREMDAFIEALG
LEEVVLVIHDWGSALGFHWAKRNPERVKGIAFMEFIRPIPTWDEW
PEFARETFQAFRTTDVGRKLIIDQNVFIEGTLPMGVVRPLTEVEM
DHYREPELNPVDREPLWREPNELPIAGEPANIVALVEEYMDWLHQ
SPVPKLLFWGTPGVLIPPAEAARLAKSLPNVKAVDIGPGLNLLQE
DNPDLIGSEIARWLSTLEISG
SEQ ID NO: 6
ATGggcAGATCTATGAAGACTATTCACGTGAGTGTCGTAACTCCC
GACGGGCCTGTATATGAGGATGACGTTGAAATGGTGAGCGTCAAA
GCAAAAAGTGGCGAGCTCGGTATTCTCCCAGGCCACATTCCCTTG
AAAGCTCCCCTAGAGATCAGTGCCGCACGCCTGAAGAAAGGGGGC
AAAACACAGtatATCGCTGTGTCAGGCGGCAACCTCGAAGTGCGG
CCTGACAAGGTGACCATCTATGCCCAGGCAGCCGAGAGGGCTGAG
GATATCGATGTCCTGCGCGCCAAGaaaGCCAAGGAGAGAGCCGAG
CGCCGACTCCAATCACAGGTCCTGGtCgggAACGTCTATATCACC
GCCGACAAGCAGAAGAACGGCATCAAGGCGAACTTCAAGATCCGC
CACAACGTGGAGGACGGCAGCaTGCAGCTCGCCGACCACTACCAG
CAGAACACCCCCATCGGCGACGGCCCCGTGCTGCTGCCCGACAAC
CACTACCTGAGCACCCAGTCCGTGCTGAGCAAAGACCCLAACGAG
AAGCGCGATCACATGGTCCTGCTGGAGTTCGTGACCGCCGCCGGG
ATCACTCTCGGCATGGACGAGCTGTACAAGGGCGGTACCGGAGGG
AGCATGAGCAAGGGCGAGGAGCTGTTCACCGGGGTGGTGCCCATC
CTGGTCGAGCTGGACGGCGACGTAAACGGCCACAAGTTCAGCGTG
CGCGGCGAGGGCGAGGGCGATGCCACCAACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCAGCTTCAAGGACGACGGC
ACCTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTTTatttgtgtgGAC
ATCGATTTTAAGCGCGCCGAGCTCgccCTTAAGCGCGCAATGAAT
AGGCTCTCAGTTGCCGAAATGAAGCTGCAGggcggaggctcgggc
ggaggtgggtcgggtggcggcggatcaatggccgagatcggcacc
ggcttccccttcgacccccactacgtggaggtgctgggcgagcgc
atgcactacgttgacgtgggcccccgcgacggcacccccgtgctg
ttcctgcacggcaaccccaccagcagctacgtgtggcgcaacatc
atcccccacgtggcccccacccaccgcactatcgcccccgacctg
atcggcatgggcaagagcgacaagcccgacctgggctacttcttc
gacgaccacgtgcgcttcatggacgccttcatcgaggccctgggc
ctggaggaggtggtgctggtgatccacgactggggcagcgccctg
ggcttccactgggccaagcgcaaccccgagcgcgtgaagggcatc
gccttcatggagttcatccgccccatccccacctgggacgagtgg
cccgagttcgcccgcgagaccttccaggccttccgcaccaccgac
gtgggccgcaagctgatcatcgaccagaacgtgttcatcgagggc
accctgcccatgggcgtggtgcgccccctgaccgaggtggagatg
gaccactaccgcgagcccttcctgaaccccgtggaccgcgagccc
ctgtggcgcttccccaacgagctgcccatcgccggcgagcccgcc
aacatcgtggccctggtggaggagtacatggactggctgcaccag
agccccgtgcccaagctgctgttctggggcacccccggcgtgctg
atcccccccgccgaggccgcccgcctggccaagagcctgcccaac
gttaaggccgtggacatcggccccggcctgaacctgctccaggag
gacaaccccgacctgatcggcagcgagatcgcccgctggctgagc
accctCgagatcagcggc
pAAV.CAG (cyto).L1-VLVG.L2-ICV.A95K
SEQ ID NO: 7
MGRSMKTIHVSVVTPDGPVYEDDVEMVSVKAKSGELGILPGHIPL
KAPLEISAARLKKGGKTQYIAVSGGNLEVRPDKVTIYAQAAERAE
DIDVLRAKKAKERAERRLQSQVLVGNVYITADKQKNGIKANFKIR
HNVEDGSMQLADHYQQNTPIGDGPVLLPDNHYLSTQSVLSKDPNE
KRDHMVLLEFVTAAGITLGMDELYKGGTGGSMSKGEELFTGVVPI
LVELDGDVNGHKESVRGEGEGDATNGKLTLKFICTTGKLPVPWPT
LVTTLTYGVQCFSRYPDHMKQHDFFKSAMPEGYVQERTISFKDDG
TYKTRAEVKFEGDTLVNRIELKGIDFKEDGNILGHKLEYNFICVD
IDEKRAELALKRAMNRLSVAEMK
SEQ ID NO: 8
ATGggcAGATCTATGAAGACTATTCACGTGAGTGTCGTAACTCCC
GACGGGCCTGTATATGAGGATGACGTTGAAATGGTGAGCGTCAAA
GCAAAAAGTGGCGAGCTCGGTATTCTCCCAGGCCACATTCCCTTG
AAAGCTCCCCTAGAGATCAGTGCCGCACGCCTGAAGAAAGGGGGC
AAAACACAGtatATCGCTGTGTCAGGCGGCAACCTCGAAGTGCGG
CCTGACAAGGTGACCATCTATGCCCAGGCAGCCGAGAGGGCTGAG
GATATCGATGTCCTGCGCGCCAAGaaaGCCAAGGAGAGAGCCGAG
CGCCGACTCCAATCACAGGTCCTGGtCgggAACGTCTATATCACC
GCCGACAAGCAGAAGAACGGCATCAAGGCGAACTTCAAGATCCGC
CACAACGTGGAGGACGGCAGCaTGCAGCTCGCCGACCACTACCAG
CAGAACACCCCCATCGGCGACGGCCCCGTGCTGCTGCCCGACAAC
CACTACCTGAGCACCCAGTCCGTGCTGAGCAAAGACCCtAACGAG
AAGCGCGATCACATGGTCCTGCTGGAGTTCGTGACCGCCGCCGGG
ATCACTCTCGGCATGGACGAGCTGTACAAGGGCGGTACCGGAGGG
AGCATGAGCAAGGGCGAGGAGCTGTTCACCGGGGTGGTGCCCATC
CTGGTCGAGCTGGACGGCGACGTAAACGGCCACAAGTTCAGCGTG
CGCGGCGAGGGCGAGGGCGATGCCACCAACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCAGCTTCAAGGACGACGGC
ACCTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTTTatttgtgtgGAC
ATCGATTTTAAGCGCGCCGAGCTCgccCTTAAGCGCGCAATGAAT
AGGCTCTCAGTTGCCGAAATGAAG
pAAV.CAG (cyto).L1-VLVG.L2-ICV.A95K.
S29W.HaloTag
SEQ ID NO: 9
MGRSMKTIHVSVVTPDGPVYEDDVEMVSVKAKWGELGILPGHIPL
KAPLEISAARLKKGGKTQYIAVSGGNLEVRPDKVTIYAQAAERAE
DIDVLRAKKAKERAERRLQSQVLVGNVYITADKQKNGIKANFKIR
HNVEDGSMQLADHYQQNTPIGDGPVLLPDNHYLSTQSVLSKDPNE
KRDHMVLLEFVTAAGITLGMDELYKGGTGGSMSKGEELFTGVVPI
LVELDGDVNGHKFSVRGEGEGDATNGKLTLKFICTTGKLPVPWPT
LVTTLTYGVQCFSRYPDHMKQHDFFKSAMPEGYVQERTISFKDDG
TYKTRAEVKFEGDTLVNRIELKGIDFKEDGNILGHKLEYNFICVD
IDEKRAELALKRAMNRLSVAEMKLQGGGSGGGGSGGGGSMAEIGT
GFPFDPHYVEVLGERMHYVDVGPRDGTPVLFLHGNPTSSYVWRNI
IPHVAPTHRTIAPDLIGMGKSDKPDLGYFFDDHVREMDAFIEALG
LEEVVLVIHDWGSALGFHWAKRNPERVKGIAFMEFIRPIPTWDEW
PEFARETFQAFRTTDVGRKLIIDQNVFIEGTLPMGVVRPLTEVEM
DHYREPFLNPVDREPLWRFPNELPIAGEPANIVALVEEYMDWLHQ
SPVPKLLFWGTPGVLIPPAEAARLAKSLPNVKAVDIGPGLNLLQE
DNPDLIGSEIARWLSTLEISG
SEQ ID NO: 10
ATGggcAGATCTATGAAGACTATTCACGTGAGTGTCGTAACTCCC
GACGGGCCTGTATATGAGGATGACGTTGAAATGGTGAGCGTCAAA
GCAAAAtggGGCGAGCTCGGTATTCTCCCAGGCCACATTCCCTTG
AAAGCTCCCCTAGAGATCAGTGCCGCACGCCTGAAGAAAGGGGGC
AAAACACAGtatATCGCTGTGTCAGGCGGCAACCTCGAAGTGCGG
CCTGACAAGGTGACCATCTATGCCCAGGCAGCCGAGAGGGCTGAG
GATATCGATGTCCTGCGCGCCAAGaaaGCCAAGGAGAGAGCCGAG
CGCCGACTCCAATCACAGGTCCTGGtCgggAACGTCTATATCACC
GCCGACAAGCAGAAGAACGGCATCAAGGCGAACTTCAAGATCCGC
CACAACGTGGAGGACGGCAGCaTGCAGCTCGCCGACCACTACCAG
CAGAACACCCCCATCGGCGACGGCCCCGTGCTGCTGCCCGACAAC
CACTACCTGAGCACCCAGTCCGTGCTGAGCAAAGACCCLAACGAG
AAGCGCGATCACATGGTCCTGCTGGAGTTCGTGACCGCCGCCGGG
ATCACTCTCGGCATGGACGAGCTGTACAAGGGCGGTACCGGAGGG
AGCATGAGCAAGGGCGAGGAGCTGTTCACCGGGGTGGTGCCCATC
CTGGTCGAGCTGGACGGCGACGTAAACGGCCACAAGTTCAGCGTG
CGCGGCGAGGGCGAGGGCGATGCCACCAACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCAGCTTCAAGGACGACGGC
ACCTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTTTatttgtgtgGAC
ATCGATTTTAAGCGCGCCGAGCTCgccCTTAAGCGCGCAATGAAT
AGGCTCTCAGTTGCCGAAATGAAGCTGCAGggcggaggctcgggc
ggaggtgggtcgggtggcggcggatcaatggccgagatcggcacc
ggcttccccttcgacccccactacgtggaggtgctgggcgagcgc
atgcactacgttgacgtgggcccccgcgacggcacccccgtgctg
ttcctgcacggcaaccccaccagcagctacgtgtggcgcaacatc
atcccccacgtggcccccacccaccgcactatcgcccccgacctg
atcggcatgggcaagagcgacaagcccgacctgggctacttcttc
gacgaccacgtgcgcttcatggacgccttcatcgaggccctgggc
ctggaggaggtggtgctggtgatccacgactggggcagcgccctg
ggcttccactgggccaagcgcaaccccgagcgcgtgaagggcatc
gccttcatggagttcatccgccccatccccacctgggacgagtgg
cccgagttcgcccgcgagaccttccaggccttccgcaccaccgac
gtgggccgcaagctgatcatcgaccagaacgtgttcatcgagggc
accctgcccatgggcgtggtgcgccccctgaccgaggtggagatg
gaccactaccgcgagcccttcctgaaccccgtggaccgcgagccc
ctgtggcgcttccccaacgagctgcccatcgccggcgagcccgcc
aacatcgtggccctggtggaggagtacatggactggctgcaccag
agccccgtgcccaagctgctgttctggggcacccccggcgtgctg
atcccccccgccgaggccgcccgcctggccaagagcctgcccaac
gttaaggccgtggacatcggccccggcctgaacctgctccaggag
gacaaccccgacctgatcggcagcgagatcgcccgctggctgagc
accctCgagatcagcggc
pAAV.CAG (cyto).L1-VLVG.L2-ICV.A95K.S29W
SEQ ID NO: 11
MGRSMKTIHVSVVTPDGPVYEDDVEMVSVKAKWGELGILPGHIPL
KAPLEISAARLKKGGKTQYIAVSGGNLEVRPDKVTIYAQAAERAE
DIDVLRAKKAKERAERRLQSQVLVGNVYITADKQKNGIKANFKIR
HNVEDGSMQLADHYQQNTPIGDGPVLLPDNHYLSTQSVLSKDPNE
KRDHMVLLEFVTAAGITLGMDELYKGGTGGSMSKGEELFTGVVPI
LVELDGDVNGHKFSVRGEGEGDATNGKLTLKFICTTGKLPVPWPT
LVTTLTYGVQCFSRYPDHMKQHDFFKSAMPEGYVQERTISFKDDG
TYKTRAEVKFEGDTLVNRIELKGIDFKEDGNILGHKLEYNFICVD
IDEKRAELALKRAMNRLSVAEMK
SEQ ID NO: 12
ATGggcAGATCTATGAAGACTATTCACGTGAGTGTCGTAACTCCC
GACGGGCCTGTATATGAGGATGACGTTGAAATGGTGAGCGTCAAA
GCAAAAtggGGCGAGCTCGGTATTCTCCCAGGCCACATTCCCTTG
AAAGCTCCCCTAGAGATCAGTGCCGCACGCCTGAAGAAAGGGGGC
AAAACACAGtatATCGCTGTGTCAGGCGGCAACCTCGAAGTGCGG
CCTGACAAGGTGACCATCTATGCCCAGGCAGCCGAGAGGGCTGAG
GATATCGATGTCCTGCGCGCCAAGaaaGCCAAGGAGAGAGCCGAG
CGCCGACTCCAATCACAGGTCCTGGtCgggAACGTCTATATCACC
GCCGACAAGCAGAAGAACGGCATCAAGGCGAACTTCAAGATCCGC
CACAACGTGGAGGACGGCAGCaTGCAGCTCGCCGACCACTACCAG
CAGAACACCCCCATCGGCGACGGCCCCGTGCTGCTGCCCGACAAC
CACTACCTGAGCACCCAGTCCGTGCTGAGCAAAGACCCLAACGAG
AAGCGCGATCACATGGTCCTGCTGGAGTTCGTGACCGCCGCCGGG
ATCACTCTCGGCATGGACGAGCTGTACAAGGGCGGTACCGGAGGG
AGCATGAGCAAGGGCGAGGAGCTGTTCACCGGGGTGGTGCCCATC
CTGGTCGAGCTGGACGGCGACGTAAACGGCCACAAGTTCAGCGTG
CGCGGCGAGGGCGAGGGCGATGCCACCAACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCAGCTTCAAGGACGACGGC
ACCTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTTTatttgtgtgGAC
ATCGATTTTAAGCGCGCCGAGCTCgccCTTAAGCGCGCAATGAAT
AGGCTCTCAGTTGCCGAAATGAAG

The sensor shown below was made from (1) insertion of circularly permuted SuperFolder GFP (cpSFGFP; italicized portion of sequence below) into the ATP-binding epsilon subunit of the ATPase from Bacillus subtilis PS 3 (non-italicized portion of sequence below) between amino acids 109 and 110; (2) subsequent mutation of the residues forming the junction between the first half of the APT-binding subunit of the ATPase and the cpSFGFP to VL VG (i.e., the QD residues of the ATP-binding unit were changed to VL, and the SH residues of the cpSFGFP were changed to VG); (3) subsequent addition of residues ICV between the end of the cpSFGFP and residue 110 of the ATP-binding unit; and (4) mutating an amino acid proximal to the ATP binding site, A119, to L. Mutation sites are shown in the sequence below with bolding and double underlining.

Additional mutations in the ATP-binding unit can be made to increase affinity: A95K and S29W.

SEQ ID NO: 13:
MKTIHVSVVTPDGPVYEDDVEMVSVKAKSGELGILPGHIPLKAPL
EISAARLKKGGKTQYIAVSGGNLEVRPDKVTIYAQAAERAEDIDV
LRAKAAKERAERRLOSQVLVG_NVYITADKOKNGIKANEKIRHNV
EDGSMQLADHYQQNTPIGDGPVLLPDNHYLSTQSVLSKDPNEKRD
HMVLLEFVTAAGITLGMDELYKGGTGGSMSKGEELFTGVVPILVE
LDGDVNGHKFSVRGEGEGDATNGKLTLKFICTTGKLPVPWPTLVT
TLTYGVOCESRYPDHMKQHDFFKSAMPEGYVQERTISFKDDGTYK
TRAEVKFEGDTLVNRIELKGIDEKEDGNILGHKLEYNFICVDIDF
KRAELLLKRAMNRLSVAEMK
SEQ ID NO: 14:
ATGAAGACTATTCACGTGAGTGTCGTAACTCCCGACGGGCCTGTA
TATGAGGATGACGTTGAAATGGTGAGCGTCAAAGCAAAAAGTGGC
GAGCTCGGTATTCTCCCAGGCCACATTCCCTTGAAAGCTCCCCTA
GAGATCAGTGCCGCACGCCTGAAGAAAGGGGGCAAAACACAGtat
ATCGCTGTGTCAGGCGGCAACCTCGAAGTGCGGCCTGACAAGGTG
ACCATCTATGCCCAGGCAGCCGAGAGGGCTGAGGATATCGATGTC
CTGCGCGCCAAGgccGCCAAGGAGAGAGCCGAGCGCCGACTCCAA
TCACAGGTCCTGGtCgggAACGTCTATATCACCGCCGACAAGCAG
AAGAACGGCATCAAGGCGAACTTCAAGATCCGCCACAACGTGGAG
GACGGCAGCaTGCAGCTCGCCGACCACTACCAGCAGAACACCCCC
ATCGGCGACGGCCCCGTGCTGCTGCCCGACAACCACTACCTGAGC
ACCCAGTCCGTGCTGAGCAAAGACCCLAACGAGAAGCGCGATCAC
ATGGTCCTGCTGGAGTTCGTGACCGCCGCCGGGATCACTCTCGGC
ATGGACGAGCTGTACAAGGGCGGTACCGGAGGGAGCATGAGCAAG
GGCGAGGAGCTGTTCACCGGGGTGGTGCCCATCCTGGTCGAGCTG
GACGGCGACGTAAACGGCCACAAGTTCAGCGTGCGCGGCGAGGGC
GAGGGCGATGCCACCAACGGCAAGCTGACCCTGAAGTTCATCTGC
ACCACCGGCAAGCTGCCCGTGCCCTGGCCCACCCTCGTGACCACC
CTGACCTACGGCGTGCAGTGCTTCAGCCGCTACCCCGACCACATG
AAGCAGCACGACTTCTTCAAGTCCGCCATGCCCGAAGGCTACGTC
CAGGAGCGCACCATCAGCTTCAAGGACGACGGCACCTACAAGACC
CGCGCCGAGGTGAAGTTCGAGGGCGACACCCTGGTGAACCGCATC
GAGCTGAAGGGCATCGACTTCAAGGAGGACGGCAACATCCTGGGG
CACAAGCTGGAGTACAACTTTatttgtgtgGACATCGATTTTAAG
CGCGCCGAGCTCttgCTTAAGCGCGCAATGAATAGGCTCTCAGTT
GCCGAAATGAAG

It would be appreciated that another protein can be added to the C-Terminus of the sensor for normalization of expression and focus artefacts. That protein can be another fluorescent protein, that is either pH-insensitive (such as mRuby3) or pH-sensitive (such as pH-mScarlet) or it can be a protein that can be specifically labeled with synthetic fluorophores, such as HaloTag or SnapTag. The synthetic fluorophores can be pH-insensitive (such as JF-X650) or pH-sensitive (such as LAMP-shade violet).

In addition to the nucleic acids and polypeptides disclosed herein (i.e., SEQ ID NOs: 2, 4, 6, 8, 10, 12 or 14, and SEQ ID NOs: 1, 3, 5, 7, 9, 11 or 13, respectively), the skilled artisan will further appreciate that changes can be introduced into a nucleic acid molecule (e.g., SEQ ID NOs: 2, 4, 6, 8, 10, 12 or 14), thereby leading to changes in the amino acid sequence of the encoded polypeptide (e.g., SEQ ID NOs: 1, 3, 5, 7, 9, 11 or 13). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5 (Suppl. 3): 345-352), which provides frequency tables for amino acid substitutions), and a non-conservative substitution is one in which an amino acid residue is replaced with an amino acid residue that does not have a similar side chain.

Also provided are nucleic acids and polypeptides that differ from SEQ ID NOs: 2, 4, 6, 8, 10, 12 or 14, and SEQ ID NOs: 1, 3, 5, 7, 9, 11 or 13, respectively. Nucleic acids and polypeptides that differ in sequence from SEQ ID NOs: 2, 4, 6, 8, 10, 12 or 14, and SEQ ID NOs: 1, 3, 5, 7, 9, 11 or 13, respectively, can have at least 95% sequence identity (e.g., at least 96%, 97%, 98%, 99% or 100% sequence identity) to SEQ ID NOs: 2, 4, 6, 8, 10, 12 or 14, and SEQ ID NOs: 1, 3, 5, 7, 9, 11 or 13, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

As used herein, an “isolated” nucleic acid molecule is a nucleic acid that is separated from other nucleic acids that are usually associated with the reference nucleic acid in the genome. Thus, an “isolated” nucleic acid includes, without limitation, a nucleic acid that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid is generally introduced into a vector (e.g., a cloning vector, or an expression vector) for convenience of manipulation or to generate a fusion nucleic acid molecule. In addition, an isolated nucleic acid can include an engineered nucleic acid molecule such as a recombinant or a synthetic nucleic acid.

Isolated nucleic acids can be obtained using techniques routine in the art. For example, isolated nucleic acids can be obtained using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Vectors containing nucleic acids that encode polypeptides also are provided. Vectors, including expression vectors, suitable for use are commercially available and/or produced by recombinant DNA technology methods routine in the art. A vector containing a nucleic acid can have elements necessary for expression operably linked to such a nucleic acid, and further can include sequences such as those encoding a selectable marker (e.g., an antibiotic resistance gene), and/or those that can be used in purification of a polypeptide (e.g., 6×His tag).

Elements necessary for expression include nucleic acid sequences that direct and regulate expression of nucleic acid coding sequences. One example of an element necessary for expression is a promoter sequence. Elements necessary for expression also can include introns, enhancer sequences, response elements, or inducible elements that modulate expression of a nucleic acid. Elements necessary for expression can be of bacterial, yeast, insect, mammalian, or viral origin and vectors can contain a combination of elements from different origins. Elements necessary for expression are described, for example, in Goeddel, 1990, Gene Expression Technology: Methods in Enzymology, 185, Academic Press, San Diego, CA. As used herein, operably linked means that a promoter and/or other regulatory element(s) are positioned in a vector relative to a nucleic acid in such a way as to direct or regulate expression of the nucleic acid.

Another aspect pertains to host cells into which a vector, e.g., an expression vector, or an isolated nucleic acid molecule has been introduced. Many methods for introducing nucleic acids into host cells, both in vivo and in vitro, are well known to those skilled in the art and include, without limitation, calcium phosphate precipitation, electroporation, heat shock, lipofection, microinjection, and viral-mediated nucleic acid transfer. The term “host cell” refers not only to the particular cell but also to the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

The term “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

Polypeptides can be purified from natural sources (e.g., a biological sample) by known methods such as DEAE ion exchange, gel filtration, and hydroxyapatite chromatography. A purified polypeptide also can be obtained, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

This disclosure also provides for articles of manufacture that can be used to detect ATP. An article of manufacture as provided herein can include any of the ATP-detecting fluorescence-based sensor described herein (e.g., nucleic acids, polypeptides, vectors and/or host cells) together with suitable packaging materials.

Articles of manufacture provided herein also can contain a package insert or package label having instructions thereon for using any of the ATP-detecting fluorescence-based sensor described herein to determine the amount of ATP in one or more cells. Articles of manufacture may additionally include reagents for carrying out the methods disclosed herein (e.g., buffers, enzymes, or co-factors).

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Mutagenesis and Screening of Bacterial Lysates

iATPSnFR1 was cloned into a bacterial expression vector derived from pRSET (Invitrogen) with restriction sites designed to make downstream subcloning into mammalian expression vectors easier. Site saturation mutagenesis was performed using the uracil template method as described previously. Mutagenesis reactions were transformed into T7 express bacterial cells (New England Biolabs) and plated on LB-Amp agar plates. Individual colonies were picked into 2 mL, square well, 96-well plates filled with 0.9 mL auto induction media and grown overnight at 30° C. with rapid shaking (350+RPM). To remove excess ATP, 96-well plates were centrifuged, pellets resuspended in Tris Buffered Saline, and re-centrifuged a total of 3 times, then frozen overnight as dry pellets. iATPSnFR variants were assayed in bacterial lysate by addition of 0.9 mL Mammalian Cell Imaging Buffer (20 mM HEPES, 119 mM NaCl, 2.5 mM KCl, 2 mM MgCl₂, 2 mM CaCl₂), pH 7.2), rapid vortexing, then pelleting by centrifugation. Clarified lysate was transferred to a black 96-well plate (Greiner). Fluorescence was measured in a plate reader (Tecan Spark) with 485 nm excitation (20 nm bandpass) and 535 nm emission (20 nm bandpass). ATP (Sigma) was added to 1 μM and fluorescence re-measured. ATP was added to 1 mM and fluorescence re-measured. Variants with responses to either concentration with high ΔF/F were carried forward with mutations of “linker 1” until one of those winners, L1-VLVG was good enough to become the template for mutations at “linker 2”.

### Example 2—Protein Expression and Purification and Characterization

iATPSnFR2 variants were transformed into E. coli BL21 (DE3) pLysS cells. Protein expression was induced by growth in 300 mL autoinduction media supplemented with 100 μg/mL ampicillin at 30° C. Proteins were purified by immobilized Ni-NTA affinity chromatography. iATPSnFR proteins were eluted with a 120-mL gradient from 0 to 200 mM imidazole. Fractions that were fluorescent were pooled and concentrated by ultrafiltration (Amicon) and then dialyzed by Slide-A-Lyzer cassette (30 kDa cutoff) in Mammalian Cell Imaging Buffer. Protein concentration was quantified by alkali denaturation and measurement of the GFP chromophore, with an extinction coefficient of 44,000 μM⁻¹ cm⁻¹ at 447 nm.

### Example 3—Synthetic Fluorophore Conjugation

Purified protein was mixed with 1.1 molar equivalents of JFX650 HaloTag Ligand and incubated at room temperature for 1 hour and then at 4° C. overnight. Excess HaloTag Ligand was removed by gel filtration on a PD-10 column (GE Life Sciences) and re-concentrated by ultrafiltration.

### Example 4—Equilibrium Measurements

All in vitro assays were performed in Mammalian Cell Imaging Buffer unless otherwise noted. iATPSnFR2 equilibrium measurements (affinity, specificity, pH) were performed with 0.2 μM protein in a Tecan Spark fluorimeter with 20 nm bandpass windows and excitation at 485 nm and emission at 535 nm.

### Example 5—Kinetic Measurements

iATPSnFR2 protein (0.2 μM) was rapidly mixed with an equal volume of stock ATP solution in an Applied Photophysics SX-20 stopped flow fluorimeter with a 490 nm LED excitation and 510 nm long pass filter for emission. Final concentration of protein was thus 0.1 μM. Concentrations of ATP listed in figures are the final concentration after mixing.

### Example 6—Cloning into Mammalian Expression Vectors

iATPSnFR2.HaloTag variants were subcloned by restriction digest into an AAV vector with a CAG promoter via BglII/PstI digest from the bacterial expression vector. The pAAV.CAG vector includes an extra serine after the initial methionine and lacks a polyhistidine tag. To target the sensor to the mitochondrial matrix, four repeats of COX8 (mito) leader sequence (SVLTPLLLRGLTGSARRLPVPRAK (SEQ ID NO:15)) separated by spacer (IHSLPPEGPW (SEQ ID NO:16)) was introduced at N-terminal of iAPTSnFR2 (A95A/A119L). HaloTag was incorporated at the N-terminal of iATPSnFR2.A95A.A119L separated by a linker (LQSTGSGNAVGQDTQER (SEQ ID NO: 17)). The plasmid was transformed into stb13 competent E. Coli and DNA was harvested from 200 mL growth media using an endotoxin free plasmid purification kit (Qiagen).

### Example 7—Rho° Cell Culture

Rho° and the parental LMTK-cells were maintained in T-75 flasks with DMEM+10% FBS+1 mM pyruvate+5 μM uridine. Two days prior to transfection, they were split into a 24-well plate (half Rho°, half LMTK) at 0.6 and 0.1 million cells per well, respectively. Cells were transfected with iATPSnFR2.HaloTag variants by lipofectamine (0.5 μg DNA+2 μL lipofectamine 2000 per well) for 4-6 hours in DMEM lacking FBS or antibiotics. Transfection media was replaced with fresh DMEM+10% FBS+penicillin/streptomycin overnight. Cells were labeled with JFX650 HaloTag ligand by adding the fluorophore to 100 nM for an hour. Cells were washed with Mammalian Cell Imaging Buffer and imaged in a Cytation 5 imaging reader (BioTek) over the course of about 2 hours. After about 20 minutes of imaging to establish a baseline fluorescence, the culture plate was removed from the instrument and buffer was replaced with the query buffer. The equilibration buffer contained 10 mM glucose, and the query buffers contained 10 mM 2dOG.

### Example 8—Rho° Cell Image Processing

Images were background subtracted and aligned for movement artefacts with the Gen5 software package that controls the Cytation 5 instrument. Images were assembled as TIF stacks and imported into ImageJ. For each well, an automated, custom script identified regions of interest (ROIs) by thresholding on the far-red channel and expanding by 2 pixels. Those ROIs were used to determine the ratio of green to red fluorescence for each well. ROIs were curated to remove those that outlined cells that detached or appeared to become spherical during the experiment.

### Example 9—Animals

All experiments involving animals were performed in accordance with protocols approved by the Weill Cornell Medicine Institutional Animal Care and Use Committee. Neurons were derived from Sprague-Dawley rats (Charles River Laboratories strain code: 001, RRID: RGD_734476) of either sex on postnatal days 0-2).

### Example 10—Neuronal Cell Culture

Primary neuronal cultures were prepared as previously described. Hippocampal CA1 to CA3 regions were dissected, dissociated, and plated onto poly-L-ornithine-coated coverslips. Plating media consisted of the minimal essential medium, 0.5% glucose, insulin (0.024 g/l), transferrin (0.1 μg/l), GlutaMAX 1%, N-21 (2%), and fetal bovine serum (10%). After 1-3 days in vitro (DIV), cells were fed and maintained in media with the following modifications: cytosine β-D-arabinofuranoside (4 μM) and FBS 5%. Cultures were incubated at 37° C. in a 95% air/5% CO2 incubator. Calcium phosphate-mediated gene transfer was performed on DIV 6-8, and neurons were used for experiments on DIV 14-21.

### Example 11—Neuronal Cell Imaging

Coverslips were loaded onto a custom chamber, mounted on a Zeiss inverted microscope and perfused at ˜100 μl min⁻¹ with Tyrode's solution containing: 119 mM NaCl, 2.5 mM KCl, 30 mM glucose, 25 mM HEPES, 2 mM CaCl₂), 2 mM MgCl₂, 50 μM DL-2-amino-5-phosphonovaleric acid (APV, AP5), 10 μM 6-cyano-7-nitroquinoxaline-2,3-dione (CNQX), adjusted to pH 7.4. Prior to each experiment, a JF-dye aliquot (JFX650 or JF635) was diluted (100 nM final) into the cell culture medium, incubated for 20 min and washed thoroughly twice (5 min each) with imaging buffer. The temperature was maintained at 37° C. with a custom-built objective heater under feedback control (Minco). Fluorescence was stimulated with OBIS 488 nm LX or OBIS 637 nm LS lasers (Coherent) passing through a laser speckle reducer (LSR 3005 at 12° diffusion angle, Optotune). Emission was acquired with a 40×, 1.3 numerical aperture oil immersion objective (Fluar, Zeiss) on an Andor iXon+Ultra 89π electron-multiplying charge-coupled device camera. Action potentials (APs) were evoked with platinum-iridium electrodes generating 1 msec pulses of an electric field of 10 V cm⁻¹ via a current stimulus isolator (A385, World Precision Instruments). Laser power at the back aperture was ˜0.32 mW for 637 nm and ˜0.52 mW for 488 nm. Laser wavelength excitation was alternated between frames during image acquisition, with an exposition of 100 msec at 1 Hz acquisition frequency, a custom-designed Arduino board coordinated AP and laser stimulation with frame acquisition.

### Example 12—Synaptic Bouton and Axonal Mitochondria Image Analysis

Time series of imaging pairs (HaloTag and iATPSnFR2) were automated split into two independent image series using a custom-written Fiji routine to facilitate analysis. ATP signals are reported as a ratio between iATPSnFR2: HaloTag (Green: Red). Images were analyzed using the ImageJ plug-in Time Series Analyzer V3 where 50 to 100 circular regions of interest (ROIs) of radius 1 μm corresponding to synaptic boutons or mitochondria expressing the Syn-iATPSnFR2.HaloTag or mito-iATPSnFR2.Halo (Halo dye positive) were selected. Image loading and posterior raw data saving were automatized using a homemade Python code for Fiji. Synaptic boutons signals were analyzed using homemade script routines in Igor-pro v 6.3.7.2 (Wavemetric, Lake Oswego, OR, USA). ATP ratio signal (Green: Red) was calculated per individual bouton, normalized to the baseline and fitted with a Boltzmann's sigmoidal function from the time that 10 μM koningic acid was administered, as follows:

$$f ( t ) = base + ( \frac{max}{1 + {e}^{4 α ( {t}_{\frac{1}{2}} - t )}} )$$

Where t_{1/2} is the t value where Y is at (base+max)/2, and α represents the rate of signal drop derived from:

$$\frac{d F}{{dt}_{t = {t}_{\frac{1}{2}}}} = α$$

For electrical activity experiments, single cell data was filtered by excluding any cell data where no ATP change was observed when cells were stimulated in presence of KA.

### Example 13—Sensor Design and In Vitro Characterization

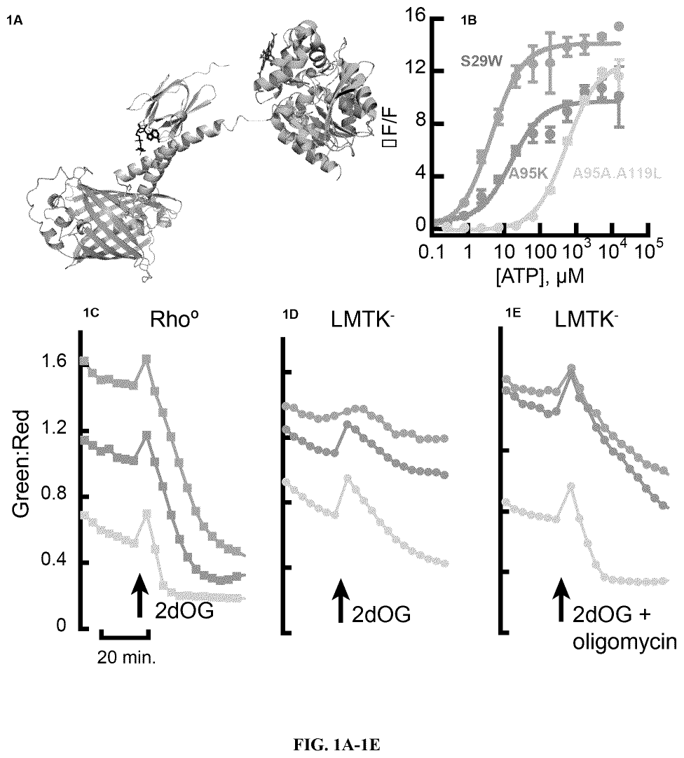

To improve upon iATPSnFR, we first re-evaluated the composition of the linkers connecting the ATP-binding domain and the cpSFGFP (FIG. 1A). This was done with the inclusion of an ATP-affinity boosting mutation, A95K, as we were also aiming to create a high-affinity ATP sensor that would be useful for the detection of sub-micromolar amounts of extracellular ATP. To increase ΔF/F, we screened thousands of variants of the sensor in bacterial lysate, as described previously, but expanded the regions mutated to cover a larger number of residues. The sensor with the highest maximum ΔF/F (saturated vs unbound) has the residues comprising “linker 1” as VLVG, where the residues QD adjacent to the ATP-binding domain are changed to VL, and the residues SH adjacent to cpSFGFP are changed to VG. Additional residues ICV were placed in “linker 2” between the end of cpSFGFP and residue 110 of the ATP-binding domain. This sensor (L1-VLVG, L2-ICV, A95K) has an affinity for ATP of ˜16 μM.

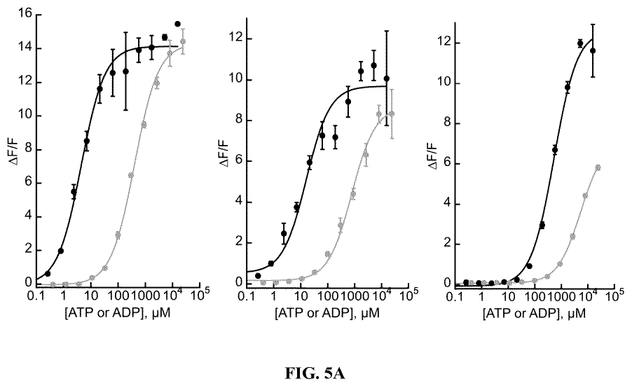

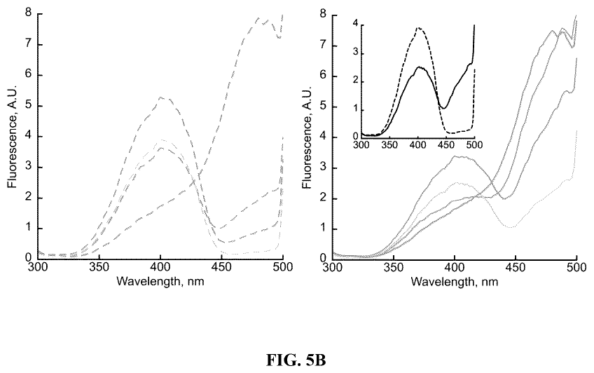

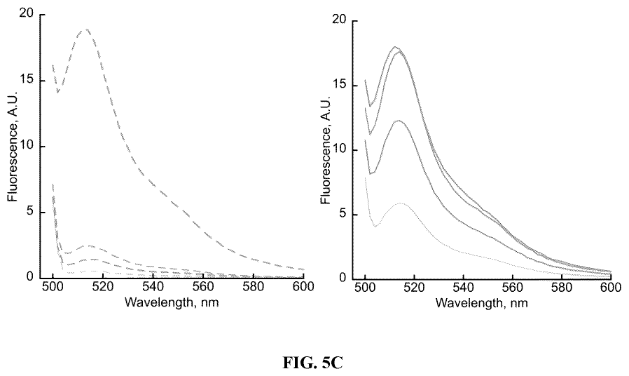

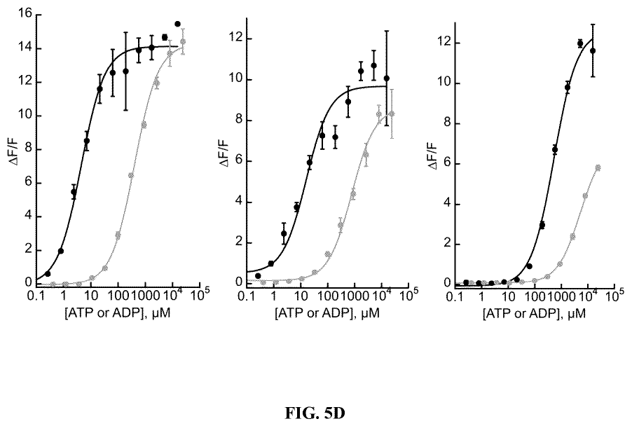

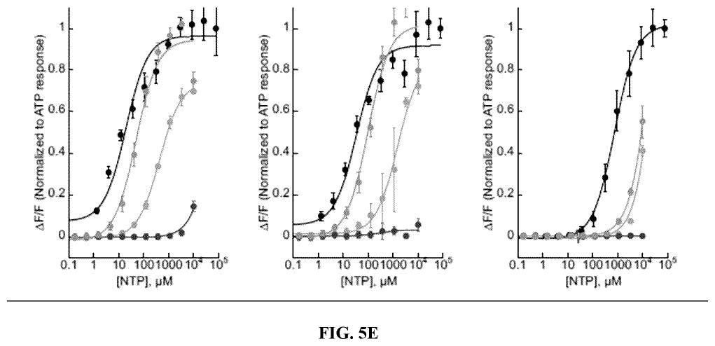

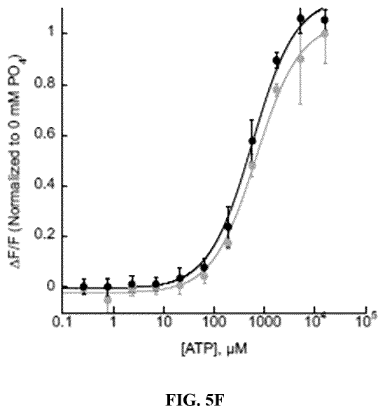

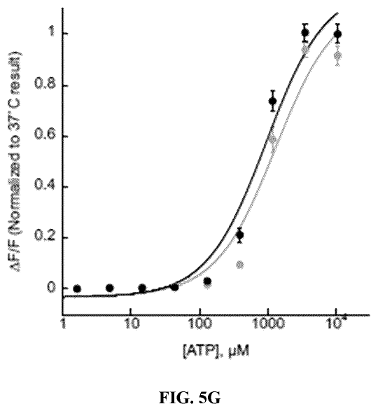

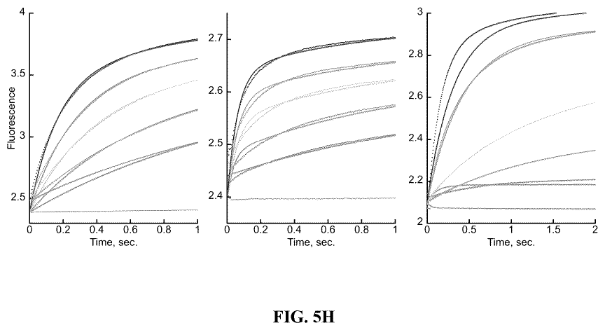

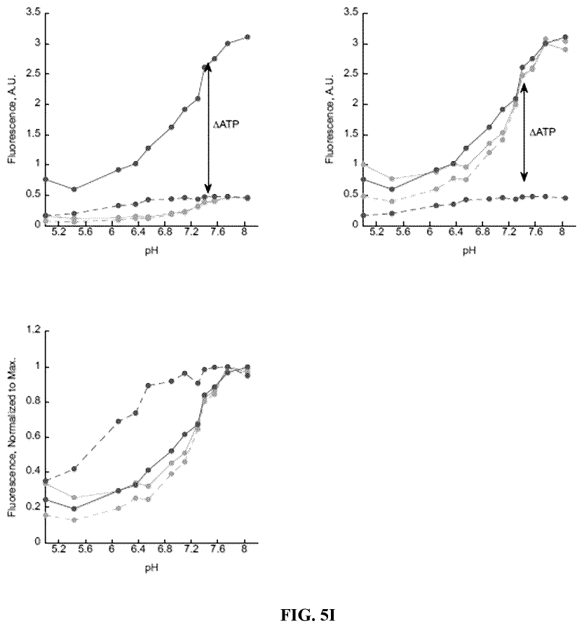

To tune the affinity of the sensor into the range needed for measuring cytosolic ATP in neurons (estimated to be ˜1.4 mM (10)), we reverted A95K back to alanine, which also reduced ΔF/F. We then screened residues near the ATP-binding site for variants that had a high ΔF/F upon saturation with ATP and had a K_{d} near 1.4 mM. The substitution A119L satisfied those two criteria and was carried forward as iATPSnFR2 (L1-VLVG, L2-ICV, A95A, A119L). In parallel, we screened other amino acid positions in the epsilon subunit in the context of A95K for variants that bound ATP more tightly. We found that the double mutant S29W.A95K has an affinity for ATP of ˜ 4 μM, giving us a trio of sensors with similar ΔF/F and varying affinities: S29W.A95K (4 μM), A95K (16 μM), A95A.A119L (530 UM). (FIG. 1B and FIG. 5A).

Like GCaMP and other cpGFP-based sensors, the excitation peaks at 405 nm and 485 nm are shifted relative to each other upon saturation with ligand, providing an isosbestic point at 436 nm (FIG. 5B, 5C), which provides an ATP-independent signal that may be useful for normalization of focus and movement artefacts. However, since imaging with shorter wavelength excitation light is often detrimental to live cells, we also developed a version of the sensor that include a C-terminal fusion proteins to act as a normalization reference. A particularly useful C-terminal fusion protein is HaloTag, which can serve as a conjugation partner for synthetic fluorophores. There are many synthetic fluorophores available for conjugation to HaloTag, and, for the present study, we primarily used JF635 or JFX650.

The iATPSnFR2.HaloTag-JFX650 was the primary protein used for in vitro characterization. The inclusion of HaloTag affects affinity, and also appears to increase the shelf-life of the purified sensor. iATPSnFR2.A95A.A119L.HaloTag-JFX650 has a ΔF/F of ˜12 and K_{d} for ATP of 500 μM (FIG. 1B), when taken at the maximum point of excitation/emission sensitivity. It has low affinity (5.3 mM) and ΔF/F (˜6) for ADP (FIG. 1B, FIG. 5D). This version of the sensor has minimal, if any, affinity for AMP. It also changes fluorescence in response to GTP and CTP, but not TTP (FIG. 5E). The affinity for these nucleoside triphosphates is low enough that their presence in cells should not affect sensor performance, given that reported concentrations of these compounds are about 0.5 mM (GTP) and 0.3 mM (CTP). Reports of other ATP sensors based on the epsilon subunit of ATPase do not mention the affinity of those sensors for CTP or TTP. MaLion and ChemoG-ATOPSIR show no change in fluorescence with GTP. Finally, iATPSnFR2 is not affected by high concentrations of inorganic phosphate (FIG. 5F).

MaLion has a markedly lower ΔF/F at 37° C. than at 25° C., and ChemoG-ATPSiR is also sensitive to temperature, but less so. In contrast, iATPSnFR2 has low temperature sensitivity, with almost identical ΔF/F and K_{d} for ATP at 37° C. and 25° C. (FIG. 5G). Moreover, in contrast to MaLion, the rate of fluorescence change for all three sensors appears to be an order of magnitude faster for iATPSnFR2 (FIG. 5H) than the published values for MaLion. Regardless, the A95K.S29W and A95K sensors bind ATP faster than the A95A.A119L variant and the rates of fluorescence increase do not follow a single exponential function (a phenomenon observed with other cpGFP-based sensors), indicating a mechanism more complicated than concerted binding and increased fluorescence. Still, the sensors reach their maximum fluorescence within 1 to 2 seconds.

Finally, since deviation from metabolic homeostasis might result in changes in the pH of the cytosol, we characterized the pH dependence of iATPSnFR2. cpSFGFP has a pKa of about 7.2 and, as expected, does not respond to ATP (FIG. 5I, green lines). The ATP-bound form of the sensor (FIG. 5J, black solid line) shows a similar pH profile, but the ligand-free sensor has a lower pKa, closer to about 6.0 (FIG. 5H, black dashed line).

### Example 14—Validation in Cell Lines

To validate the utility of iATPSnFR2 (and its affinity derivatives, A95K and A95K.S29W) for measuring changes in ATP production or consumption, we first tested in two related cell lines, LMTK-, a mouse fibroblast cell line, and a Rho° (ρ⁰) derivative, which lacks mitochondrial DNA observing changes in fluorescence in response to treatment with different ATP-affecting drugs. Because most cell lines are heavily dependent on glycolysis for energy production, we expected a significant decrease in fluorescence upon treatment with 2-deoxyglucose (2dOG). 2dOG can be phosphorylated by hexokinase, the first enzymatic step in glycolysis, but its product (2dOG-P) cannot be further processed, resulting in competitive inhibition of phosphoglucose isomerase and, therefore, intracellular ATP depletion.

Prior to treatment, the green: red ratio of imaged cells was greater when the cells expressed higher affinity variants of iATPSnFR2, indicating that affinity affected ATP saturation. (A95K.S29W>A95K>A95A.A119L. FIG. 1). When glucose was replaced with 2dOG, green fluorescence rapidly dropped in the ρ⁰ cells (FIG. 1C), which have no alternative source of ATP other than glycolysis, with the weakest affinity variant (A95A.A119L) becoming nearly non-fluorescent within minutes of treatment, followed by the next highest affinity variant (A95K) and finally, the highest affinity variant (A95K.S29W). Meanwhile, 2dOG treatment resulted in decreased fluorescence only in the parental LMTK-cells expressing the weakest affinity variant (A95A.A119L), indicating that alternative methods for generating ATP, namely through mitochondrial oxidative phosphorylation, were available (FIG. 1D). When the parent cells were treated with both 2dOG and oligomycin (to inhibit oxidative phosphorylation), the fluorescence of the parent cells dropped in a manner similar to the ρ⁰ cells (FIG. 1E).

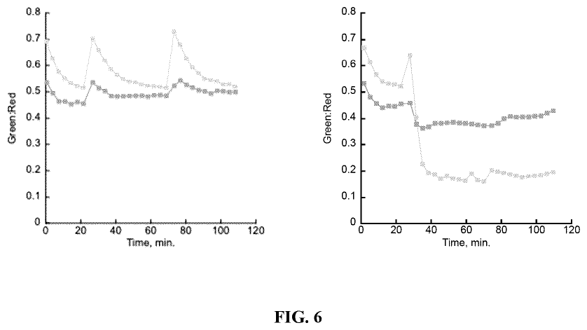

The self-consistent result of the three affinity variants responding to 2dOG as expected indicates that the primary effect of the treatment is due to decreasing [ATP], and not some other metabolic change, such as pH, [ADP] etc. To further corroborate that, we also used cpSFGFP (without an ATP binding subunit) as a “null” sensor. Since cpSFGFP and iATPSnFR2 have very similar pH dependencies, the former should provide an appropriate reporter for how treatment might affect intracellular pH or other artefactual changes in fluorescence. In ρ⁰ cells expressing either cpSFGFP or iATPSnFR2.A95A.A119L, replacement of buffer containing 10 mM glucose with fresh buffer (also containing 10 mM glucose), causes a transient increase in the green fluorescence, although the increase is greater for cells expressing iATPSnFR2 than cpSFGFP (FIG. 6), indicating that addition of fresh glucose probably gives the cells a small boost in ATP production. (Note: a transient apparent change in [ATP] upon treatment is also observed with the ChemoG-ATPSiR sensor. When the buffer is replaced by 10 mM 2dOG and later rescued with 10 mM glucose (FIG. 6), only cells expressing iATPSnFR2 show a significant decrease in fluorescence, while those expressing cpSFGFP remain mostly constant, confirming that iATPSnFR2 is primarily reporting Δ[ATP], not ΔpH.

### Example 15—Use of iATPSnFR2 to Monitor Consumption of ATP in Primary Neurons

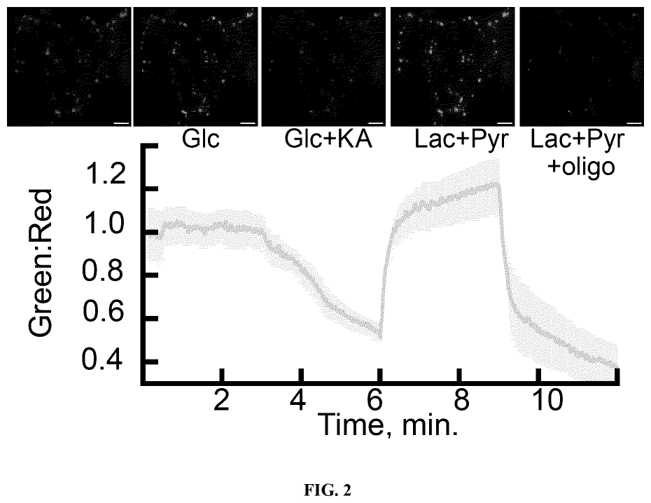

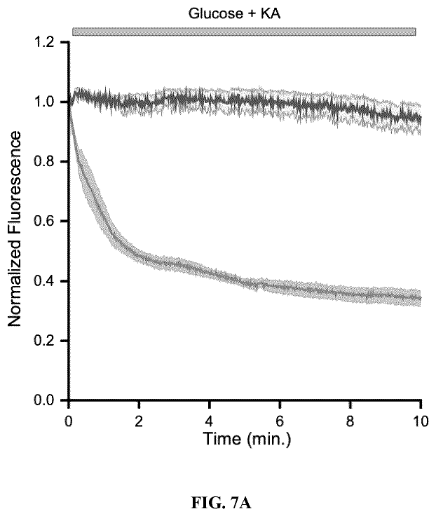

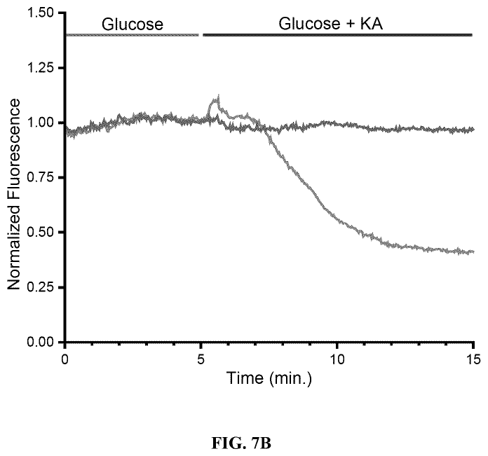

Neurons are highly polarized cells that consist of soma, axons, and dendrites. The generation and consumption of ATP in these different sub-compartments are also semi-autonomously regulated. Genetically encoded sensors can be targeted to specific subcellular compartments by using different signal sequences and can be used to monitor [ATP] during metabolic perturbations. To that end, we fused four copies of the COX8 leader sequence to the N-terminus of iATPSnFR2.A95A.A119L.HaloTag (mito-iATPSnFR2.HaloTag) to target it to the matrix of mitochondria. Expressing the targeted sensor in primary hippocampal neurons led to a distinct punctate appearance in both the red (here visualized with JF635 liganded to HaloTag) and green channels (FIG. 2), consistent with mitochondrial targeting. Application koningic acid (KA), a covalent inhibitor of glyceraldehyde 3-phosphate dehydrogenase (GAPDH) blocked glycolysis and therefore, resulted in a gradual decrease of [ATP] (FIG. 2), while JF635 signal remained unperturbed (FIG. 7A). Bath application of a mixture of lactate and pyruvate fully restored the ATP signal, as expected since this allows it to bypass the glycolytic block, directly fueling the mitochondrial tricarboxylic acid cycle. Subsequently blocking the mitochondrial F₀-F₁ ATPase with oligomycin led to a further collapse of the ATP signal (FIG. 2). Similar results were obtained using mito-iATPSnFR2.mIRFP670nano3 (FIG. 7B).

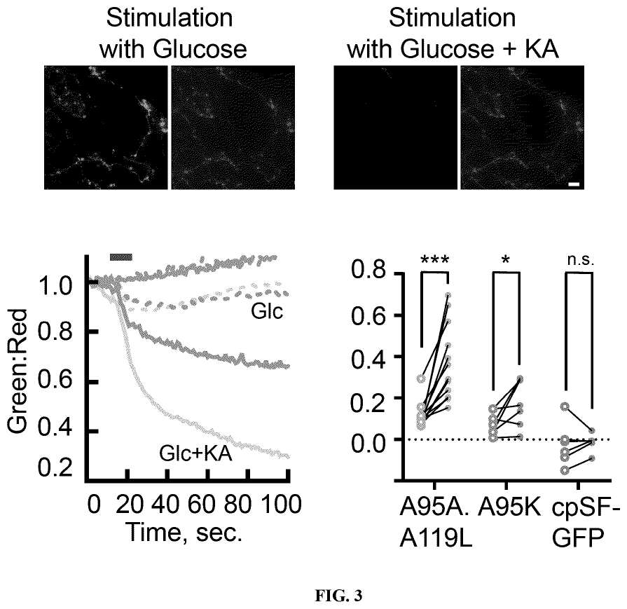

One key question concerning the biology of energy consumption in neurons is the nature of the mechanisms that maintain the balance between ATP consumption and ATP production during electrical activity. While this has previously been addressed by our use of luciferase, the time resolution of that experiment was on the order of one minute. Here we used iATPSnFR2.A95A.A119L.HaloTag-JFX650 to observe ATP dynamics with sub-second time resolution, during a 6-second window of intense action potential firing at nerve terminals of dissociated hippocampal neurons in culture (FIG. 3). When we performed the stimulation under normal conditions, fluorescence dropped slightly, and recovered to baseline within one minute of concluding the stimulation. When we subsequently inhibited production of ATP by glycolysis with KA, we observed an even greater decrease in fluorescence that did not recover within the minute and a half observation window. The use of the higher affinity iATPSnFR2.A95K.HaloTag-JFX650 was still able to detect the activity changes in fluorescence signal, albeit with a much smaller amplitude. The negative control cpSFGFP (without an ATP-binding domain) remained unchanged even after ATP depletion. These data recapitulate our previous observations that nerve terminals have a robust on-demand ATP synthesis program that responds rapidly to electrical activity, while improving the time resolution by an order of magnitude.

### Example 16—iATPSnFR2 Reveals Differences in ATP Consumption Among Individual Boutons

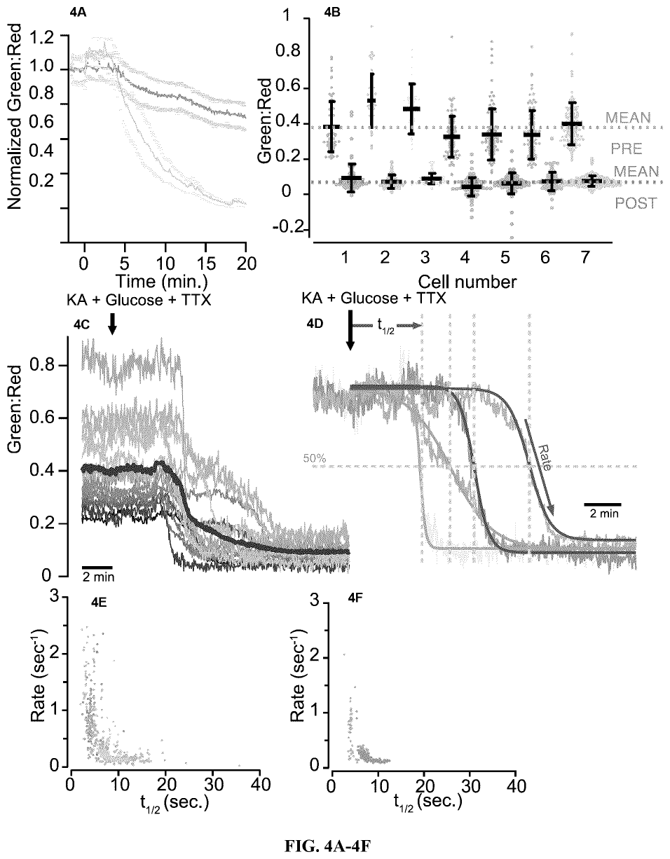

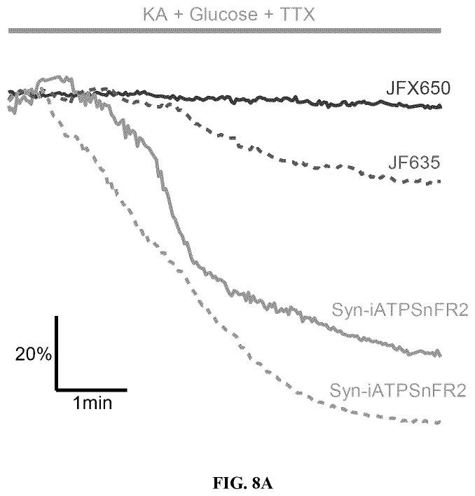

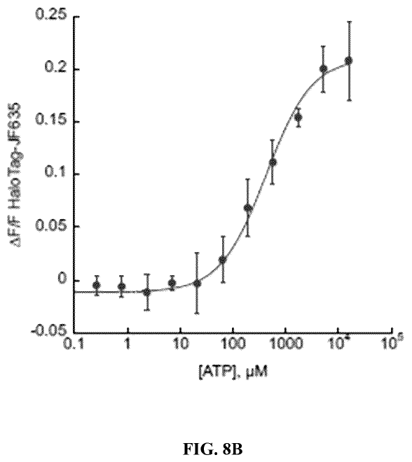

We previously showed that, even in the absence of electrical activity, nerve terminals have high basal ATP consumption but both single bouton and kinetic details were limited by the low photon flux of the luciferase-based reporter. We repeated these experiments using synaptically targeted iATPSnFR2 that was fused to synaptophysin, sending it to the synaptic vesicle surface, facing the cytosol. Similar to previous findings, when ATP production was blocked (in this case by inhibiting GAPDH with KA) in the presence of the Na⁺ channel blocker tetrodotoxin (TTX), the average green to red fluorescence ratio of iATPSnFR2.HaloTag-JFX650 (averaged over 7 different neurons, each contributing 20-30 boutons) gradually declined over a 5-10 min period as previously reported. The weaker A95A.A119L variant showed a greater drop than the medium affinity A95K variant (FIG. 4A). In contrast to what we observed in the mitochondrially-targeted sensor, when using JF635 as a HaloTag ligand, the red channel fluorescence was not inert with respect to ATP changes (FIG. 8A). Rhodamine-based dyes can interact with ATP on their own, and it appears that HaloTag-JF635 itself is sensitive to ATP, albeit with low ΔF/F (FIG. 8B). This confounding artefact was minimized by using a newer generation HaloTag dye, JFX650 (FIG. 8A). The improved signal to noise properties of iATPSnFR2 allowed us to extract several new features regarding single synapse ATP control. Comparisons of the initial ratio values across synapses and experiments showed that across boutons, the resting ATP value varied as much as 4-fold, and by 50% across cells, but this variation was minimized following ATP depletion by KA (FIG. 4B). Similarly, the kinetics of ATP depletion in individual nerve terminals behaved unexpectedly. Although the average behavior across all boutons shows a gradual decline in ATP (FIG. 4A), no individual bouton looked like the average, displaying instead two distinct kinetic features that varied widely across boutons (FIG. 4C). At the single bouton level, ATP signals declined very rapidly (typically showing depletion within ˜ 10-20 sec) but only after a delay time that varied significantly across boutons. Individual bouton ATP levels under these conditions were best described by a simple Boltzmann function, F (t)=F0/(1+e^{4α(t-t}^{_{1/2}}⁾) where t_{1/2} describes the delay time to the precipitous drop in signal at a rate a (FIG. 4D). Both these kinetic parameters, t_{1/2} and α varied significantly across the population of nerve terminals. A correlation analysis of the two extracted parameters showed that, in general, smaller t_{1/2} were associated with higher values of a (FIG. 4E) and vice versa. Some cells showed tighter clustering of rate and t_{1/2} parameters than others (FIG. 4F).

### Example 17—Phosphoglycerate Kinase is a Central Leverage Point in Parkinson's Disease Driven Neuronal Metabolic Deficits

### ATPsnfr Analysis

For synapto-iATPSnFR2-miRFP670nano3 experiments, nerve terminals were selected in the miRFP670nano3 channels, blind to the ATPsnfr channel and then background subtracted. The traces are reported as iATPsnfr2 to miRFP670nano3 ratio, Fratio. Cell data was filtered by removing any cells that exhibited no change to activity.

### A Suppressor Screen of Hypometabolic Synaptic Failure Identifies PGK1 as a Rate Limiting Enzyme in Presynaptic Bioenergetics

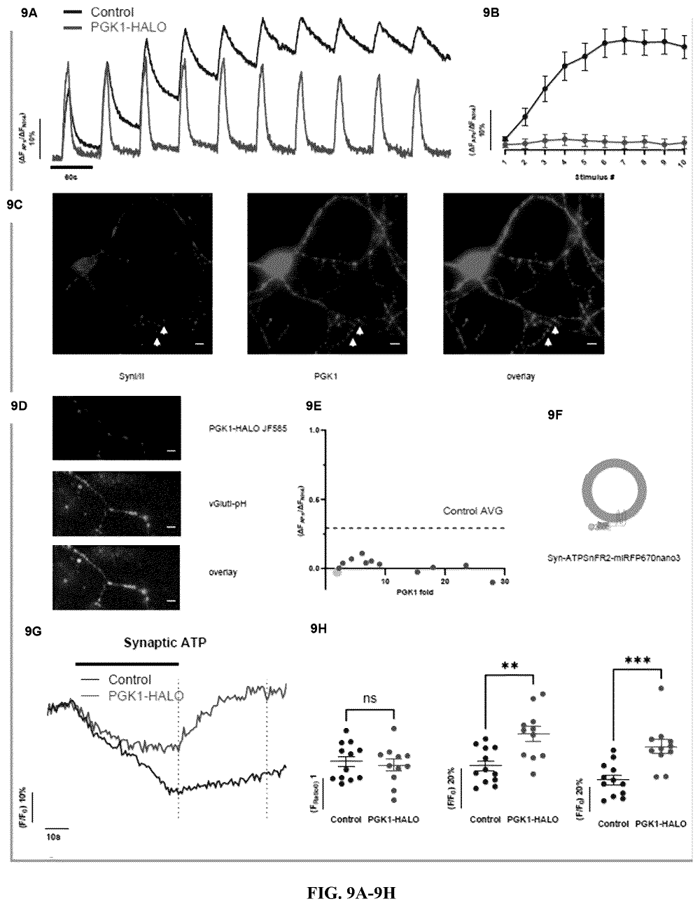

The gradual synaptic failure under hypometabolic conditions allowed us to conduct genetic expression suppressor screen of glycolytic enzymes to see if any single one, when over-expressed, would allow synapses to sustain function under limited fuel availability. Of the enzymes evaluated, only one, PGK1, conferred significant hypometabolic resilience. PGK1, which, when overexpressed, was able to fully support synaptic function in 0.1 mM glucose, exhibiting robust SV recycling for all ten rounds of activity tested (FIG. 9A, 9B). This functional rescue was conducted using a HALO-tagged PGK1, allowing us to visualize the expressed protein distribution in live cells, confirming that the functional rescue was neuron specific. Quantitative immunofluorescence for PGK1 and synapsin showed that native PGK1, although expressed throughout the cell, was highly concentrated in nerve terminals (FIG. 9C). Quantitative comparison of presynaptic PGK1 immunofluorescence showed that, on average, our expression construct led to a ˜9-fold over-expression of this enzyme at nerve terminals (compared to non-transfected cells) that varied significantly across experiments. Live cell labeling of the HALO moiety (FIG. 9D) allowed us to determine, on a cell-by-cell basis, the presynaptic abundance of PGK1-HALO and determine quantitatively how exogenous PGK1-HALO expression correlated with hypometabolic resilience (FIG. 9E). These data revealed that every cell overexpressing PGK1-HALO was able to confer metabolic resilience, even at the lowest expression level achieved, ˜ 2-fold. As expected, SV recycling was impaired in neurons expressing an shRNA targeting PGK1.

The dramatic impact of PGK1 expression on hypometabolic synapse function strongly implies that increasing this enzyme's abundance leads to a robust change in the kinetics of presynaptic ATP synthesis. To test this idea, we carried out parallel measurements of presynaptic ATP dynamics using a next-generation genetically encoded ratiometric ATP reporter (synapto-iATPSnFR2.0-miRFP670nano3) targeted to nerve terminals 21 (FIG. 9F). Under hypometabolic conditions, a sustained 60 s burst of 600 AP led to a 30% depletion of presynaptic ATP that only recovered minimally for the next 30 s (FIG. 9G, 9H). In neurons expressing PGK1-HALO, the initial pre-stimulus ATP values were similar to controls, however the decrease in ATP during the stimulus was blunted (decreasing by 14%), while in contrast to controls, ATP levels recovered rapidly over the next 30s. These data demonstrate that increased PGK1 abundance is sufficient to increase the kinetics of ATP production during demand, indicating that, following activity, PGK1 is a rate-limiting enzyme in nerve terminal glycolysis and that a modest change in abundance has dramatic functional consequences.

### Terazosin Restores Synaptic Function Under Hypometabolic Conditions

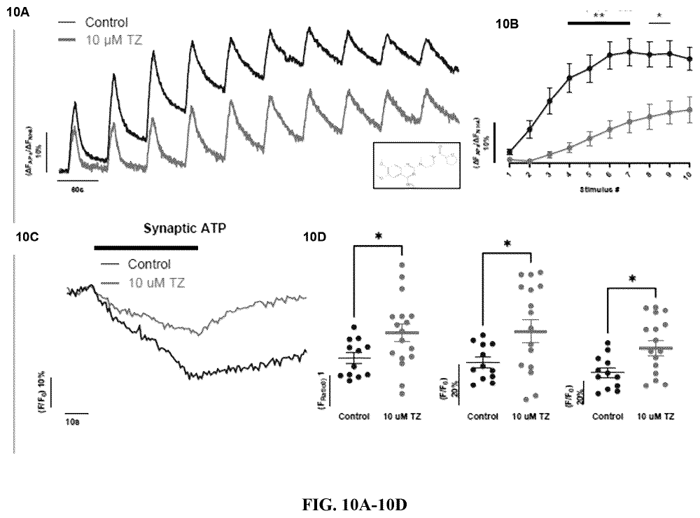

As one of the starting points for implicating a pivotal role for PGK1 in PD was the identification of PGK1 as an off-target binder of TZ, we examined whether TZ mimicked PGK1 expression in our synaptic endurance paradigm. Treating neurons with 10 μM TZ conferred significant resilience to stimulation in low glucose (FIG. 10A, 10B) albeit to a lesser extent than with PGK1 expression. This impact was eliminated in neurons where PGK1 expression was depleted by expression of an shRNA targeting PGK1. Consistent with in vitro enzymology 10, a lower dose (1 μM) was ineffective, while a higher dose (100 μM) was either ineffective or likely deleterious, since we found few cells that survived this treatment. TZ's impact on synaptic metabolism was also independent of the known clinical target of the drug, the α1 adrenergic receptor (α1R), as a modified version of TZ that lacks potency for α1R, TZ-md23, was equally effective at conferring synaptic endurance. Parallel ATP measurements showed that TZ lead to improved nerve terminal ATP production as well. In neurons treated with TZ, resting ATP levels were slightly elevated, ATP depletion during strong stimulation in low glucose was blunted, and the recovery following such a stimulus was accelerated (FIG. 10C, 10D) compared to controls. These data collectively support the concept that PGK1 under low glucose conditions is rate-limiting for ATP production at nerve terminals, and that TZ increases PGK1 activity and thereby ATP production.

PARK7 (DJ-1) Interacts with PGK1 and is Necessary for PGK1 Mediated Synaptic Resilience

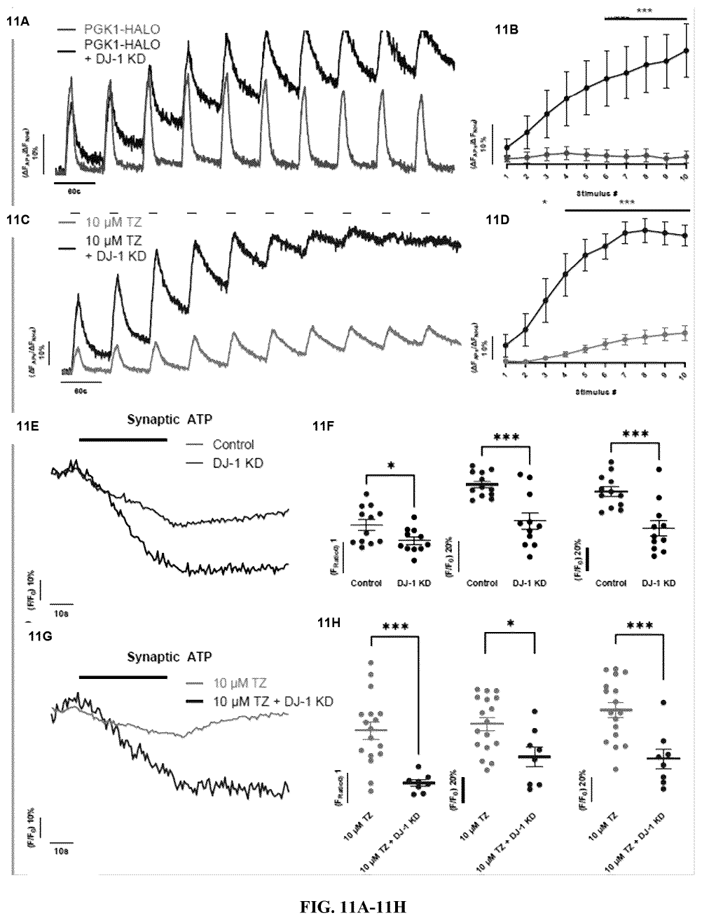

An unbiased chemical screen recently led to the identification of an interaction between PGK1 and DJ-1, the product of the PARK7 gene, a genetic driver of familial PD. DJ-1 is a chaperone with strong structurally similarity to HSP31 but the precise clientele that it supports related to PD is poorly understood. To investigate whether DJ-1 has a functional impact on PGK1 function, we probed PGK1 rescue of hypometabolic synaptic function, but in absence of DJ-1. Although shRNA-mediated loss of DJ-1 led to a very similar gradual slowing of SV recycling in low glucose under repeated stimulation to that seen in control neurons over-expression of PGK1 or incubation with TZ in absence of DJ-1 completely failed to restore synaptic endurance under low glucose conditions (FIG. 11A, 11B, 11C, 11D). The need for DJ-1 to confer PGK1-dependent metabolic resilience strongly suggests, that loss of DJ-1 might itself impact synaptic ATP dynamics during activity. To test this idea, we carried out measurements of presynaptic ATP dynamics in DJ-1 KD neurons using synapto-iATPSnFR2.0-miRFP670nano3. These experiments revealed a striking inability of DJ-1 KD nerve terminals to sustain ATP production during stimulation under hypometabolic conditions (FIG. 11E). During prolonged stimulation in low glucose, ATP levels dropped by 60%, more than double that in control neurons and exhibited no recovery thereafter (FIG. 11F). Consistent with the inability of PGK1 activity enhancement to rescue hypometabolic synapse function, TZ application showed no acceleration of ATP production in DJ-1 KD neurons following a prolonged burst of activity (FIG. 11G, 11H). The impairment in ATP production resulting from loss of DJ-1, was likely due to a loss of glycolytic function and not mitochondrial ATP production as a slowing of synaptic vesicle recycling in low glucose and during mild stimulation was only apparent when we additionally blocked mitochondrial ATP production with the F1-F0 ATPase inhibitor, oligomycin. Furthermore, when glucose was substituted with a mixture of lactate and pyruvate, SV recycling kinetics in DJ-1 KD neurons was indistinguishable from control. As expected from the impact on ATP production due to the loss of DJ-1, SV recycling in DJ-1 KD neurons was dramatically slowed following intense stimulation. This slowing of SV recycling was reversed upon re-expression of an shRNA-insensitive DJ-1 variant but could not be reversed by PGK1 expression or incubation with TZ.

### Example 18—DDHD2 is Necessary for Activity-Driven Fatty Acid Fueling of Nerve Terminal Function

Fatty Acids Derived from Axonal LDs Drive Beta-Oxidation and Axonal Mitochondrial ATP Production

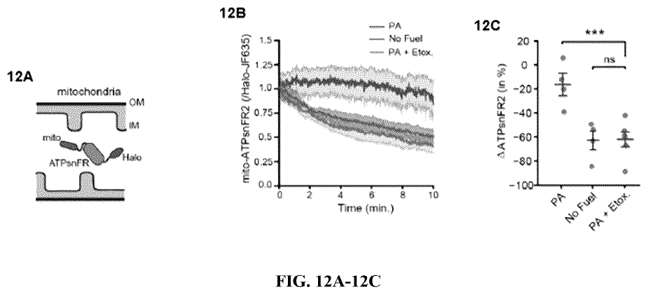

We made use of a newly developed large dynamic range genetically encoded ATP sensor targeted to the mitochondrial matrix, mito-iATPSnFR218, expressed in primary neurons to examine ATP production in the mitochondrial matrix (FIG. 12A) under different fuel conditions. Following acute removal of glucose, axonal mitochondrial ATP declines slowly for the next 10 minutes, as expected since pyruvate production via glycolysis should be largely absent. However, in neurons that had been pre-fed with palmitic acid for 4 hours mitochondrial ATP levels were sustained over the same period in the complete absence of glucose. In contrast if the CPT1 inhibitor, etomoxir was applied at the same time as removing glucose, the decline in mitochondrial ATP level in palmitic acid pre-fed neurons was identical to the control neurons without any fuel supplement (FIG. 12B, 12C). Similar results were obtained in neurons where LDs were induced by blocking DDHD2 activity with KLH45 for 24 hours. When glucose and KLH45 are removed, ATP levels are sustained, but including etomoxir under these conditions lead to ATP decline over a 10-minute period. These experiments demonstrate that axonal mitochondria can sustain mitochondrial ATP production using FAs derived from LDs in the complete absence of glucose.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. An ATP-detecting fluorescence-based sensor having at least 95% sequence identity to an amino acid sequence selected from SEQ ID NOs: 1, 3, 5, 7, 9, 11, and 13.

2. The ATP-detecting fluorescence-based sensor of claim 1, having at least 96% sequence identity to an amino acid sequence selected from SEQ ID NOs: 1, 3, 5, 7, 9, 11, and 13.

3. The ATP-detecting fluorescence-based sensor of claim 1, having at least 97% sequence identity to an amino acid sequence selected from SEQ ID NOs: 1, 3, 5, 7, 9, 11, and 13.

4. The ATP-detecting fluorescence-based sensor of claim 1, having at least 98% sequence identity to an amino acid sequence selected from SEQ ID NOs: 1, 3, 5, 7, 9, 11, and 13.

5. The ATP-detecting fluorescence-based sensor of claim 1, having at least 99% sequence identity to an amino acid sequence selected from SEQ ID NOs: 1, 3, 5, 7, 9, 11, and 13.

6. The ATP-detecting fluorescence-based sensor of claim 1, having 100% sequence identity to an amino acid sequence selected from SEQ ID NOs: 1, 3, 5, 7, 9, 11, and 13.

7. An ATP-detecting fluorescence-based sensor having at least 95% sequence identity to the amino acid sequence shown in SEQ ID NO: 13 and including at least A95K and S29W substitutions.

8. An ATP-detecting fluorescence-based sensor having at least 95% sequence identity to the amino acid sequence shown in SEQ ID NO:13 and including at least an A95K substitution.

9. An ATP-detecting fluorescence-based sensor having at least 95% sequence identity to the amino acid sequence shown in SEQ ID NO: 13 and including at least A95A and A119L substitutions.

10. A nucleic acid encoding the ATP-detecting fluorescence-based sensor of claim 1.

11. The nucleic acid of claim 10, wherein the nucleic acid comprises at least 95% sequence identity to a nucleic acid sequence selected from SEQ ID NOs: 2, 4, 6, 8, 10, 12 and 14.

12. A vector comprising the nucleic acid of claim 10.

13. A cell comprising the vector of claim 12.

14. A method of determining the amount of ATP in a cell, comprising:
providing a cell comprising the ATP-detecting fluorescence-based sensor of claim 1, and
determining the amount of fluorescence emitted from the sensor.

15. The method of claim 14, further comprising exposing the cell to ATP or a source thereof.

16. The method of claim 14, wherein the cell is in vitro.

17. The method of claim 14, wherein the cell is in vivo.

18. An article of manufacture, comprising the ATP-detecting fluorescence-based sensor of claim 1.

19. The article of manufacture of claim 18, further comprising ATP.

20. The article of manufacture of claim 18, further comprising an ATP inhibitor.

21. The article of manufacture of claim 18, further comprising instructions for using the ATP-detecting fluorescence-based sensor.


SEQUENCE LISTING (17 sequences)

SEQ ID NO: 1 - protein, 383 aa, synthetic construct
MGRSMKTIHVSVVTPDGPVYEDDVEMVSVKAKSGELGILPGHIPLKAPLEISAARLKKGGKTQYIAVSGGNLEVRPDKVTIYAQAAERAEDIDVLRAKAAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELLLKRAMNRLSVAEMK

SEQ ID NO: 2 - DNA, 1149 bp, synthetic construct
  source 1..1149: mol_type = other DNA
atgggcagatctatgaagactattcacgtgagtgtcgtaactcccgacgggcctgtatatgaggatgacgttgaaatggtgagcgtcaaagcaaaaagtggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaaggccgccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcttgcttaagcgcgcaatgaataggctctcagttgccgaaatgaag

SEQ ID NO: 3 - protein, 696 aa, synthetic construct
MGRSMKTIHVSVVTPDGPVYEDDVEMVSVKAKSGELGILPGHIPLKAPLEISAARLKKGGKTQYIAVSGGNLEVRPDKVTIYAQAAERAEDIDVLRAKAAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELLLKRAMNRLSVAEMKLQGGGSGGGGSGGGGSMAEIGTGFPFDPHYVEVLGERMHYVDVGPRDGTPVLFLHGNPTSSYVWRNIIPHVAPTHRTIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNVKAVDIGPGLNLLQEDNPDLIGSEIARWLSTLEISG

SEQ ID NO: 4 - DNA, 2088 bp, synthetic construct
  source 1..2088: mol_type = other DNA
atgggcagatctatgaagactattcacgtgagtgtcgtaactcccgacgggcctgtatatgaggatgacgttgaaatggtgagcgtcaaagcaaaaagtggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaaggccgccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcttgcttaagcgcgcaatgaataggctctcagttgccgaaatgaagctgcagggcggaggctcgggcggaggtgggtcgggtggcggcggatcaatggccgagatcggcaccggcttccccttcgacccccactacgtggaggtgctgggcgagcgcatgcactacgttgacgtgggcccccgcgacggcacccccgtgctgttcctgcacggcaaccccaccagcagctacgtgtggcgcaacatcatcccccacgtggcccccacccaccgcactatcgcccccgacctgatcggcatgggcaagagcgacaagcccgacctgggctacttcttcgacgaccacgtgcgcttcatggacgccttcatcgaggccctgggcctggaggaggtggtgctggtgatccacgactggggcagcgccctgggcttccactgggccaagcgcaaccccgagcgcgtgaagggcatcgccttcatggagttcatccgccccatccccacctgggacgagtggcccgagttcgcccgcgagaccttccaggccttccgcaccaccgacgtgggccgcaagctgatcatcgaccagaacgtgttcatcgagggcaccctgcccatgggcgtggtgcgccccctgaccgaggtggagatggaccactaccgcgagcccttcctgaaccccgtggaccgcgagcccctgtggcgcttccccaacgagctgcccatcgccggcgagcccgccaacatcgtggccctggtggaggagtacatggactggctgcaccagagccccgtgcccaagctgctgttctggggcacccccggcgtgctgatcccccccgccgaggccgcccgcctggccaagagcctgcccaacgttaaggccgtggacatcggccccggcctgaacctgctccaggaggacaaccccgacctgatcggcagcgagatcgcccgctggctgagcaccctcgagatcagcggc

SEQ ID NO: 5 - protein, 696 aa, synthetic construct
MGRSMKTIHVSVVTPDGPVYEDDVEMVSVKAKSGELGILPGHIPLKAPLEISAARLKKGGKTQYIAVSGGNLEVRPDKVTIYAQAAERAEDIDVLRAKKAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELALKRAMNRLSVAEMKLQGGGSGGGGSGGGGSMAEIGTGFPFDPHYVEVLGERMHYVDVGPRDGTPVLFLHGNPTSSYVWRNIIPHVAPTHRTIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNVKAVDIGPGLNLLQEDNPDLIGSEIARWLSTLEISG

SEQ ID NO: 6 - DNA, 2088 bp, synthetic construct
  source 1..2088: mol_type = other DNA
atgggcagatctatgaagactattcacgtgagtgtcgtaactcccgacgggcctgtatatgaggatgacgttgaaatggtgagcgtcaaagcaaaaagtggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaagaaagccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcgcccttaagcgcgcaatgaataggctctcagttgccgaaatgaagctgcagggcggaggctcgggcggaggtgggtcgggtggcggcggatcaatggccgagatcggcaccggcttccccttcgacccccactacgtggaggtgctgggcgagcgcatgcactacgttgacgtgggcccccgcgacggcacccccgtgctgttcctgcacggcaaccccaccagcagctacgtgtggcgcaacatcatcccccacgtggcccccacccaccgcactatcgcccccgacctgatcggcatgggcaagagcgacaagcccgacctgggctacttcttcgacgaccacgtgcgcttcatggacgccttcatcgaggccctgggcctggaggaggtggtgctggtgatccacgactggggcagcgccctgggcttccactgggccaagcgcaaccccgagcgcgtgaagggcatcgccttcatggagttcatccgccccatccccacctgggacgagtggcccgagttcgcccgcgagaccttccaggccttccgcaccaccgacgtgggccgcaagctgatcatcgaccagaacgtgttcatcgagggcaccctgcccatgggcgtggtgcgccccctgaccgaggtggagatggaccactaccgcgagcccttcctgaaccccgtggaccgcgagcccctgtggcgcttccccaacgagctgcccatcgccggcgagcccgccaacatcgtggccctggtggaggagtacatggactggctgcaccagagccccgtgcccaagctgctgttctggggcacccccggcgtgctgatcccccccgccgaggccgcccgcctggccaagagcctgcccaacgttaaggccgtggacatcggccccggcctgaacctgctccaggaggacaaccccgacctgatcggcagcgagatcgcccgctggctgagcaccctcgagatcagcggc

SEQ ID NO: 7 - protein, 383 aa, synthetic construct
MGRSMKTIHVSVVTPDGPVYEDDVEMVSVKAKSGELGILPGHIPLKAPLEISAARLKKGGKTQYIAVSGGNLEVRPDKVTIYAQAAERAEDIDVLRAKKAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELALKRAMNRLSVAEMK

SEQ ID NO: 8 - DNA, 1149 bp, synthetic construct
  source 1..1149: mol_type = other DNA
atgggcagatctatgaagactattcacgtgagtgtcgtaactcccgacgggcctgtatatgaggatgacgttgaaatggtgagcgtcaaagcaaaaagtggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaagaaagccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcgcccttaagcgcgcaatgaataggctctcagttgccgaaatgaag

SEQ ID NO: 9 - protein, 696 aa, synthetic construct
MGRSMKTIHVSVVTPDGPVYEDDVEMVSVKAKWGELGILPGHIPLKAPLEISAARLKKGGKTQYIAVSGGNLEVRPDKVTIYAQAAERAEDIDVLRAKKAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELALKRAMNRLSVAEMKLQGGGSGGGGSGGGGSMAEIGTGFPFDPHYVEVLGERMHYVDVGPRDGTPVLFLHGNPTSSYVWRNIIPHVAPTHRTIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNVKAVDIGPGLNLLQEDNPDLIGSEIARWLSTLEISG

SEQ ID NO: 10 - DNA, 2088 bp, synthetic construct
  source 1..2088: mol_type = other DNA
atgggcagatctatgaagactattcacgtgagtgtcgtaactcccgacgggcctgtatatgaggatgacgttgaaatggtgagcgtcaaagcaaaatggggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaagaaagccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcgcccttaagcgcgcaatgaataggctctcagttgccgaaatgaagctgcagggcggaggctcgggcggaggtgggtcgggtggcggcggatcaatggccgagatcggcaccggcttccccttcgacccccactacgtggaggtgctgggcgagcgcatgcactacgttgacgtgggcccccgcgacggcacccccgtgctgttcctgcacggcaaccccaccagcagctacgtgtggcgcaacatcatcccccacgtggcccccacccaccgcactatcgcccccgacctgatcggcatgggcaagagcgacaagcccgacctgggctacttcttcgacgaccacgtgcgcttcatggacgccttcatcgaggccctgggcctggaggaggtggtgctggtgatccacgactggggcagcgccctgggcttccactgggccaagcgcaaccccgagcgcgtgaagggcatcgccttcatggagttcatccgccccatccccacctgggacgagtggcccgagttcgcccgcgagaccttccaggccttccgcaccaccgacgtgggccgcaagctgatcatcgaccagaacgtgttcatcgagggcaccctgcccatgggcgtggtgcgccccctgaccgaggtggagatggaccactaccgcgagcccttcctgaaccccgtggaccgcgagcccctgtggcgcttccccaacgagctgcccatcgccggcgagcccgccaacatcgtggccctggtggaggagtacatggactggctgcaccagagccccgtgcccaagctgctgttctggggcacccccggcgtgctgatcccccccgccgaggccgcccgcctggccaagagcctgcccaacgttaaggccgtggacatcggccccggcctgaacctgctccaggaggacaaccccgacctgatcggcagcgagatcgcccgctggctgagcaccctcgagatcagcggc

SEQ ID NO: 11 - protein, 383 aa, synthetic construct
MGRSMKTIHVSVVTPDGPVYEDDVEMVSVKAKWGELGILPGHIPLKAPLEISAARLKKGGKTQYIAVSGGNLEVRPDKVTIYAQAAERAEDIDVLRAKKAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELALKRAMNRLSVAEMK

SEQ ID NO: 12 - DNA, 1149 bp, synthetic construct
  source 1..1149: mol_type = other DNA
atgggcagatctatgaagactattcacgtgagtgtcgtaactcccgacgggcctgtatatgaggatgacgttgaaatggtgagcgtcaaagcaaaatggggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaagaaagccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcgcccttaagcgcgcaatgaataggctctcagttgccgaaatgaag

SEQ ID NO: 13 - protein, 379 aa, synthetic construct
MKTIHVSVVTPDGPVYEDDVEMVSVKAKSGELGILPGHIPLKAPLEISAARLKKGGKTQYIAVSGGNLEVRPDKVTIYAQAAERAEDIDVLRAKAAKERAERRLQSQVLVGNVYITADKQKNGIKANFKIRHNVEDGSMQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFICVDIDFKRAELLLKRAMNRLSVAEMK

SEQ ID NO: 14 - DNA, 1137 bp, synthetic construct
  source 1..1137: mol_type = other DNA
atgaagactattcacgtgagtgtcgtaactcccgacgggcctgtatatgaggatgacgttgaaatggtgagcgtcaaagcaaaaagtggcgagctcggtattctcccaggccacattcccttgaaagctcccctagagatcagtgccgcacgcctgaagaaagggggcaaaacacagtatatcgctgtgtcaggcggcaacctcgaagtgcggcctgacaaggtgaccatctatgcccaggcagccgagagggctgaggatatcgatgtcctgcgcgccaaggccgccaaggagagagccgagcgccgactccaatcacaggtcctggtcgggaacgtctatatcaccgccgacaagcagaagaacggcatcaaggcgaacttcaagatccgccacaacgtggaggacggcagcatgcagctcgccgaccactaccagcagaacacccccatcggcgacggccccgtgctgctgcccgacaaccactacctgagcacccagtccgtgctgagcaaagaccctaacgagaagcgcgatcacatggtcctgctggagttcgtgaccgccgccgggatcactctcggcatggacgagctgtacaagggcggtaccggagggagcatgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaactttatttgtgtggacatcgattttaagcgcgccgagctcttgcttaagcgcgcaatgaataggctctcagttgccgaaatgaag

SEQ ID NO: 15 - protein, 24 aa, synthetic construct
SVLTPLLLRGLTGSARRLPVPRAK

SEQ ID NO: 16 - protein, 10 aa, synthetic construct
IHSLPPEGPW

SEQ ID NO: 17 - protein, 17 aa, synthetic construct
LQSTGSGNAVGQDTQER